(19) United States
(12) Patent Application Publication
(10) Pub. No.: US 2023/0174626 A1
(43) Pub. Date: Jun. 8, 2023

(54) ANTIBODIES

(71) Applicant: GLAXOSMITHKLINE BIOLOGICALS SA, Rixensart (BE)

(72) Inventors: Marco BIANCUCCI, Rockville, MD (US); Sumana CHANDRAMOULI, Rockville, MD (US); Kate LUISI, Rockville, MD (US)

(73) Assignee: GLAXOSMITHKLINE BIOLOGICALS SA, Rixensart (BE)

(21) Appl. No.: 17/913,264

(22) PCT Filed: Mar. 25, 2021
(86) PCT No.: PCT/IB2021/052482

§ 371 Date: Sep. 21, 2022

Related U.S. Application Data
(60) Provisional application No. 63/000,578, filed on Mar. 27, 2020.
(60) Provisional application No. 63/007,566, filed on Apr. 9, 2020.

(51) Int. Cl. C07K 16/08; G01N 33/569

(52) U.S. Cl. CPC C07K 16/088; G01N 33/56994; C07K 2317/24; C07K 2317/565

(57) ABSTRACT

Monoclonal antibodies and antigen binding fragments that specifically bind to the Human Cytomegalovirus (HCMV) pentamer protein complex (gH/gL/UL128/UL130/UL131A) or other gH-containing complexes, and uses thereof.

### FIELD

The present invention relates to monoclonal antibodies and antigen binding fragments thereof that bind to the Human Cytomegalovirus (HCMV) pentamer protein complex, or other gH-containing protein complexes such as gH/gL and gH/gL/gO, and uses of the same.

### BACKGROUND

Human cytomegalovirus (HCMV) is a leading viral cause of birth defects and solid organ transplant rejection (see, e.g., Boppana et al., Clin Infect Dis. 2013;57 Suppl 4:S178-81; Britt, Curr Top Microbiol Immunol. 2008;325:417-70). For individuals with a compromised immune system, such as a person with HIV infection or transplant recipients, HCMV can cause life-threatening illnesses and/or transplant failure. In addition, HCMV infection has been associated with numerous inflammatory diseases, cancers, and cardiovascular pathologies.

HCMV is a betaherpesvirus capable of broad cell tropism by utilizing several envelope glycoprotein complexes to attach to, and fuse with, host cell membranes. HCMV glycoprotein B (gB) is a class III viral fusion protein. The natural conformation of HCMV gB within the viral envelope is a homotrimer. Trimeric gb, with the complex of HCMV glycoprotein H (gH) and glycoprotein L (gL) (gH/gL complex), forms the core fusion machinery conserved among all herpesvirus (see, e.g., Connolly et al., Nat Rev Microbiol. 2011;9(5):369-81). While gB catalyzes membrane fusion between virus and infected cells, two gH/gL-containing complexes regulate viral tropism: the trimer of glycoproteins gH, gL and gO (gH/gL/gO) and the pentamer of gH, gL, and proteins UL128, UL130, UL131A (Pentamer gH/gL/UL128/UL130/UL131A, also referred to as gH/gL/UL128-131) (see, e.g., Ciferri et al., Proc Natl Acad Sci U S A. 2015;112(6): 1767-72; Vanarsdall et al., Proc Natl Acad Sci U S A. 2019;116(9):3728-33.

In particular, gH/gL/gO is primarily required for viral entry on fibroblasts through platelet-derived growth factor receptor alpha (PDGFR-a), although recent studies have suggested that gH/gL/gO might be required for entry on all cell types (see, e.g., Kabanova et al., Proc Natl Acad Sci U S A. 2014;111(50): 17965-70; Stegmann et al., PLoS Pathog. 2017;13(4):e1006273; Wu et al., PLoS Pathog. 2017;13(4):e1006281; Gerna et al., J Virol. 2016;90(14):6216-23; Zhou et al. J Virol. 2015;89(17):8999-9009.

HCMV Pentamer binds to Neuropilin-2 receptor (NRP2) to mediate HCMV entry in epithelial, endothelial, myeloid and other cell types, but not fibroblasts. See e.g., Gerna et al., J Gen Virol. 2008;89(Pt 4):853-65; Wang et al., Proc Natl Acad Sci U S A. 2005;102(50): 18153-8; Ryckman et al., J Virol. 2006;80(2):710-22; Martinez-Martin et al, Cell. 2018;174(5):1158-71 e19.

The majority of neutralizing antibodies found in human sera against HCMV infection of epithelial cells target the Pentamer protein complex and assist in preventing infection in epithelial and endothelial cells (see, e.g., Fouts et al., J Virol. 2012;86(13):7444-7). Pentamer-specific antibodies are several orders of magnitude more potent than other HCMV glycoprotein antibodies (see, e.g., Chiuppesi et al., J Virol. 2015;89(23):11884-98; Freed et al., PNAS 2013;110(51):E4997-E5005; Macagno et al., J Virol. 2010;84(2): 1005-13. Epub 2009/11/06). Pentamer antibodies may block primary infection in human placental cytotrophoblast; early response against Pentamer in seronegative women with primary infection during pregnancy is positively correlated with a lower risk of transmission of virus to the fetus (see, e.g., Lilleri et al., PLoS One (2013) 8(3):e59863). Pentamer-based vaccines have been reported to elicit strong and broadly neutralizing responses in animal models (see, e.g., Freed et al., PNAS 2013;110(51):E4997-E5005; Wang et al., Sci Transl Med. 2016;8(362):362ra145; Wen et al., Vaccine. 2014;32(30):3796-804; Wussow et al., J Virol. 2013;87(3): 1322-32; John et al., Vaccine. 2018;36(12): 1689-99).

Vaccine antigens incorporating gH/gL have also been reported as raising an immune response against fibroblast entry, increasing the breadth of the immune response (Wen et al., Vaccine. 2014;32(30):3796-804).

Monoclonal antibodies (mAbs) and antigen binding fragments thereof useful for the detection and characterization of HCMV Pentamer and other gH-containing protein complexes, epitopes therein, and for use as therapeutic antibodies against HCMV, are desirable.

### SUMMARY OF THE INVENTION

In a first aspect the present invention relates to an isolated monoclonal antibody or antigen binding fragment thereof that specifically binds to HCMV Pentamer.

A further aspect of the present invention is compositions comprising a pharmaceutically acceptable carrier and an isolated monoclonal antibody or antigen binding fragment thereof that specifically binds to HCMV Pentamer.

A further aspect of the present invention is an isolated nucleic acid molecule encoding the heavy or light chain variable domain of an isolated monoclonal antibody or antigen binding fragment thereof that specifically binds to HCMV Pentamer.

A further aspect of the present invention is a method of detecting the presence of HCMV Pentamer in a sample, by contacting the sample with an effective amount of a monoclonal antibody or antigen binding fragment that specifically binds to HCMV Pentamer, under conditions sufficient to form an immune complex, and detecting the presence or absence of the immune complex.

### BRIEF DESCRIPTION OF THE DRAWINGS

FIG. 1 displays the results of epitope binning; the table cells in bold indicate 100% competition for the same neutralizing epitopes.

FIG. 2 graphs the results of binding experiments performed by Surface Plasmon Resonance (SPR). The graph bar indicates the affinity (K_{D}) of mAb 23C2 (white bar), mAb 26B6 (stippled bar), and mAb 2C7 (lined bar) for Pentamer and gH/gL/gO. Each bar represents the average of three experiments.

FIG. 3 graphs the results of competition assays with Pentamer receptor NRP-2 and mAbs that bind to Pentamer. The bar graph indicates the percentage of binding of NRP-2 in presence of the indicated mAb, where each bar represents the average of two independent experiments.

### DETAILED DESCRIPTION

### Introduction

The present inventors investigated HCMV Pentamer- and gH-specific monoclonal antibodies (mAbs).

Monoclonal antibodies elicited by mouse immunization with HCMV Pentamer were investigated for HCMV Pentamer and gH/gL/gO binding and HCMV neutralization. Three murine mAbs with high HCMV neutralization were selected and humanized. The humanized mAbs were investigated using epitope binning, antigen binding, pentamer-receptor competition and HCMV neutralization assays. These three humanized mAbs target diverse epitopes on Pentamer, and can be used as analytical tools to monitor antigen integrity, to better understand mechanisms of antibody-mediated neutralization of HCMV, and potentially as therapeutic mAbs against HCMV.

Previous structural and functional studies indicated that the most potent Pentamer antibodies target UL proteins, while less potent antibodies are directed to gH/gL complex (see, e.g., Freed et al., PNAS 2013;110(51):E4997-E5005; Macagno et al., J Virol. 2010;84(2):1005-13. Epub 2009/11/06; Chandramouli et al., Sci Immunol. 2017;2(12); Ciferri et al., PLoS Pathog. 2015;11(10); Ha et al., J Virol. 2017;91(7); Gardner et al., Nat Commun. 2016;7:13627). Seven neutralizing epitopes had originally been reported, spanning across UL surfaces; five of which are considered non-overlapping based on structural and biochemical characterization (see e.g., Macagno et al., J Virol. 2010, 84(2):1005-13; Chandramouli et al., Sci Immunol. 2017, 2(12); Ciferri et al., PLoS Pathog. 2015, 11(10)). Neutralizing epitopes 1, 2, 4/6, and 5 overlap with Pentamer-receptor binding site, as antibodies targeting these epitopes inhibit NRP2-Pentamer interaction; while site 3/7 antibodies may function by preventing the interaction of Pentamer with gB, thereby neutralizing HCMV entry by blocking activation of membrane fusion (see, e.g., Martinez-Martin, Cell (2018):174(5):1158-71; Chandramouli et al., Sci Immunol. 2017;2(12)).

Three potent HCMV neutralizing monoclonal antibodies were isolated from Pentamer immunized mice (referred to herein as m26B6 mAb, m23C2 mAb, and m2C7 mAb). The heavy and light chain variable domains of these antibodies were sequenced and used to construct humanized antibodies (indicated herein as 26B6 mAb, 23C2 mAb, and 2C7 mAb; or alternatively, as h26B6 mAb, h23C2 mAb, and h2C7 mAb, respectively). The 26B6 mAb competed with a Pentamer antibody known to target site 1 (mAb 15D8, see U.S. Pat. No. 8,287,870), while 23C2 and 2C7 mAbs did not compete with any antibody tested. These mAbs (and antigen-binding fragments thereof) may be (a) employed as analytical tools to identify epitopes present on Pentamer antigens, constructs and fragments, (b) used to better understand Pentamer function and role in viral replication and antibody mediated neutralization of HCMV, and/or (c) used in immunotherapy against HCMV infection.

The mAbs 23C2 and 2C7 revealed two previously uncharacterized epitopes on Pentamer. The 26B6 mAb competes with Site 1 mAb 15D8, see e.g., Macagno et al., J Virol 84:1005-1013 (2010)). All three mAbs are highly neutralizing in epithelial cells. 2C7 is neutralizing in fibroblasts.

The sequences of the heavy and light chain variable domains (VH and VL, respectively) for mAbs 26B6, 23C2, and 2C7 were determined and are provided herein as SEQ ID NOs: 1-6. Binding of an antibody to an epitope of an antigen is primarily directed by the VH and VL Complementarity Determining Regions (CDRs). The amino acid sequence of CDRs within a VH or VL can be determined using methods as are known in the art. See e.g., Kabat et al., Sequences of Proteins of Immunological Interest, 5th Ed. Public Health Service, National Institutes of Health, Bethesda, MD, 1991, regarding the “Kabat” numbering scheme; Al-Lazikani et al., J. Mol. Biol. 273(4):927-948 1997, regarding the “Chothia” numbering scheme; Lefranc et al., Dev. Comp. Immunol. 27:55-77, 2003, regarding “IMGT” numbering scheme. Paratome is an automated tool that identifies antigen binding regions (ABRs) that are similar to CDRs in that they function in antibody-antigen binding (see e.g., Kunik, PLoS Comput Biol 8(2):e1002388, published online 2012 Feb 23, doi: 10.1371/journal.pcbi.1002388); and Kunik et al., Nucleic Acids Res. 2012 July 40 (Web server issue): W521-4, doi: 10.1093/nar/gks480; program available on the internet at ofranservices.biu.ac.il/index.html. The Paratome ABRs and IMGT, Kabat, and Chothia CDRs partially overlap. As used herein, and unless specified otherwise, the term “CDR” encompasses CDRs identified by any method, including the Kabat, IMGT and Chothia methods, and additionally encompasses the Paratome ABRs.

The “Kabat” CDRs of the heavy chain variable domain of mAb 26B6 (SEQ ID NO: 1) as determined using tools available online at www.abysis.org are provided in Table 1.

TABLE 1

| Region | Sequence | Residues | Length |
|---|---|---|---|
| Leader | MEFGLSWVFL VAILEGVHC (SEQ ID NO:25) | 1-19 | 19 |
| HFR1 | EVQLVESGGG LVKPGGSLKL SCAASGFTIT (SEQ ID NO: 28) | 20-49 | 30 |
| HCDR1 | DYYIY (SEQ ID NO:7) | 50-54 | 5 |
| HFR2 | WLRQTPEKKL EWVA (SEQ ID NO: 29) | 55-68 | 14 |
| HCDR2 | AISDGGRYTD YPDSVKG (SEQ ID NO: 8) | 69-85 | 17 |
| HFR3 | RFTMSSDNAK TNLYLQMGSL QSDDTGMYYC AR (SEQ ID NO: 30) | 86-117 | 32 |
| HCDR3 | WASYGYHWYF DV (SEQ ID NO: 9) | 118-129 | 12 |
| HFR4 | WGAGTTVTVS S (SEQ ID NO: 26) | 130-140 | 11 |
| Tail | ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKRVEP KSCDKTHTCP PCPAPELLGG PSVFLFPPKP KDTLMISRTP EVTCVVVDVS HEDPEVKFNW YVDGVEVHNA KTKPREEQYN STYRWSVLT VLHQDWLNGK EYKCKVSNKA LPAPIEKTIS KAKGQPREPQ VYTLPPSREE MTKNQVSLTC LVKGFYPSDI AVEWESNGQP ENNYKTTPPV LDSDGSFFLY SKLTVDKSRW QQGNVFSCSV MHEALHNHYT QKSLSLSPGK (SEQ ID NO: 27) | 141-470 | 330 |

HFR = Heavy chain Framework Region

The “Kabat” CDRs of the light chain variable domain of mAb 26B6 (SEQ ID NO:2) as determined using tools available online at www.abysis.org are provided in Table 2.

TABLE 2

| Region | Sequence | Residues | Length |
|---|---|---|---|
| Leader | METPAELLFL LLLWLPDTTG (SEQ ID NO: 31) | 1-20 | 20 |
| LFR1 | DIQMTQTTSS LSASLGDRVT ISC (SEQ ID NO: 32) | 21-43 | 23 |
| LCDR1 | SASQDISNYI N (SEQ ID NO: 10) | 44-54 | 11 |
| LFR2 | WYQQKRDGTV KLLIY (SEQ ID NO: 33) | 55-69 | 15 |
| LCDR2 | DSSSLHS (SEQ ID NO: 11) | 70-76 | 7 |
| LFR3 | GVPSRFSGSG SGTDYSLTIS NLEPEDFATY FC (SEQ ID NO: 34) | 77-108 | 32 |
| LCDR3 | QQYSNLPPT (SEQ ID NO: 12) | 109-117 | 9 |
| LFR4 | FGGGTNLEIK RTV (SEQ ID NO: 35) | 118-130 | 13 |
| Tail | AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC (SEQ ID NO: 36) | 131-234 | 104 |

LFR = Light chain Framework Region

The “Kabat” CDRs of the heavy chain variable domain of mAb 23C2 (SEQ ID NO:3) as determined using tools available online at www.abysis.org are provided in Table 3.

TABLE 3

| Region | Sequence | Residues | Length |
|---|---|---|---|
| Leader | MEFGLSWVFL VAILEGVHC (SEQ ID NO: 25) | 1-19 | 19 |
| HFR1 | DVQLQESGPG LVKPSQSLSL TCSVTGYSIK (SEQ ID NO: 37) | 20-49 | 30 |
| HCDR1 | SGYYWN (SEQ ID NO: 13) | 50-55 | 6 |
| HFR2 | WIRQFPGNKL EWMG (SEQ ID NO: 38) | 56-69 | 14 |
| HCDR2 | YISYGGDFTY NPSLKN (SEQ ID NO: 14) | 70-85 | 16 |
| HFR3 | RISITRDTSK NQFFLKLNSV TTEDTATYFC VR (SEQ ID NO: 39) | 86-117 | 32 |
| HCDR3 | EPNWESYFDI (SEQ ID NO: 15) | 118-127 | 10 |
| HFR4 | WGAGTTVTVS S (SEQ ID NO: 26) | 128-138 | 11 |
| Tail | ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKRVEP KSCDKTHTCP PCPAPELLGG PSVFLFPPKP KDTLMISRTP EVTCVVVDVS HEDPEVKFNW YVDGVEVHNA KTKPREEQYN STYRWSVLT VLHQDWLNGK EYKCKVSNKA LPAPIEKTIS KAKGQPREPQ VYTLPPSREE MTKNQVSLTC LVKGFYPSDI AVEWESNGQP ENNYKTTPPV LDSDGSFFLY SKLTVDKSRW QQGNVFSCSV MHEALHNHYT QKSLSLSPGK (SEQ ID NO: 27) | 139-468 | 330 |

HFR = Heavy chain Framework Region

The “Kabat” CDRs of the light chain variable domain of mAb 23C2 (SEQ ID NO:4) as determined using tools available online at www.abysis.org are provided in Table 4.

TABLE 4

| Region | Sequence | Residues | Length |
|---|---|---|---|
| Leader | METPAELLFL LLLWLPDTTG (SEQ ID NO: 40) | 1-20 | 20 |
| LFR1 | DIQMTQSPAS LSASVGETVT ITC (SEQ ID NO: 41) | 21-43 | 23 |
| LCDR1 | RASDNIYSYL T (SEQ ID NO: 16) | 44-54 | 11 |
| LFR2 | WYHQKQGKSP HLLVY (SEQ ID NO: 42) | 55-69 | 15 |
| LCDR2 | NAKTLPE (SEQ ID NO: 17) | 70-76 | 7 |
| LFR3 | GVPLRFSGTG SGTQFSLKIN SLQPEDFGIY FC (SEQ ID NO: 43) | 77-108 | 32 |
| LCDR3 | LHDYGFPRT (SEQ ID NO: 18) | 109-117 | 9 |
| LFR4 | FGGGTILEVK RTV (SEQ ID NO: 44) | 118-130 | 13 |
| Tail | AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC (SEQ ID NO: 45) | 131-234 | 104 |

LFR = Light chain Framework Region

The “Kabat” CDRs of the heavy chain variable domain of mAb 2C7 (SEQ ID NO:5) as determined using tools available online at www.abysis.org are provided in Table 5.

TABLE 5

| Region | Sequence | Residues | Length |
|---|---|---|---|
| Leader | MEFGLSWVFL VAILEGVHC (SEQ ID NO: 25) | 1-19 | 19 |
| HFR1 | EVQLQQSGPE LEKPGASVKI SCKASGYSFT (SEQ ID NO: 46) | 20-49 | 30 |
| HCDR1 | GYNMN (SEQ ID NO: 19) | 50-54 | 5 |
| HFR2 | WVKQSNGKSL EWIG (SEQ ID NO: 47) | 55-68 | 14 |
| HCDR2 | NIDPYFGGTS YNQKFKG (SEQ ID NO: 20) | 69-85 | 17 |
| HFR3 | KATLTVDKSS STAYMELKSL TSEDSAVYYC AR (SEQ ID NO: 48) | 86-117 | 32 |
| HCDR3 | HYGYFPWFAY (SEQ ID NO: 21) | 118-127 | 10 |
| HFR4 | WGQGTLVTVS A (SEQ ID NO: 49) | 128-138 | 11 |
| Tail | ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKRVEP KSCDKTHTCP PCPAPELLGG PSVFLFPPKP KDTLMISRTP EVTCVVVDVS HEDPEVKFNW YVDGVEVHNA KTKPREEQYN STYRWSVLT VLHQDWLNGK EYKCKVSNKA LPAPIEKTIS KAKGQPREPQ VYTLPPSREE MTKNQVSLTC LVKGFYPSDI AVEWESNGQP ENNYKTTPPV LDSDGSFFLY SKLTVDKSRW QQGNVFSCSV MHEALHNHYT QKSLSLSPGK (SEQ ID NO: 27) | 139-468 | 330 |

HFR = Heavy chain Framework Region

The “Kabat” CDRs of the light chain variable domain of mAb 2C7 (SEQ ID NO:6) as determined using tools available online at www.abysis.org are provided in Table 6.

TABLE 6

| Region | Sequence | Residues | Length |
|---|---|---|---|
| Leader | METPAELLFL LLLWLPDTTG (SEQ ID NO: 50) | 1-20 | 20 |
| LFR1 | DIVLTQSPAS LAVSLGQRAT ISC (SEQ ID NO: 51) | 21-43 | 23 |
| LCDR1 | RASESVDSSG NSFMH (SEQ ID NO: 22) | 44-58 | 15 |
| LFR2 | WYQQKPGQPP KLLIY (SEQ ID NO: 52) | 59-73 | 15 |
| LCDR2 | RASNLES (SEQ ID NO:23) | 74-80 | 7 |
| LFR3 | GIPARFSGSG SRTDFTLTIN PVGADDVATY YC (SEQ ID NO: 53) | 81-112 | 32 |
| LCDR3 | QQSNEDPWT (SEQ ID NO: 24) | 113-121 | 9 |
| LFR4 | FGGGTKLEI KRTV (SEQ ID NO: 54) | 122-134 | 13 |
| Tail | AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC (SEQ ID NO: 55) | 135-238 | 104 |

LFR = Light chain Framework Region

Methods of using these antibodies or antibody fragments thereof to detect HCMV Pentamer or other gH-containing protein complexes are also provided herein, such as for the detection and/or quantification of HCMV Pentamer gH-containing protein complexes in a sample, such as a biological sample obtained from an individual subject. Methods of administering these antibodies to a subject to prevent or treat HCMV infection in said subject are also described. Stated another way, the antibodies of the present invention may be administered to a subject to prevent or reduce cell-to-cell spread of HCMV, and/or reduce HCMV entry in epithelial, endothelial and/or myeloid cells.

The present mAbs may also be used for epitope identification and in characterizing antigen structure, such as by hydrogen deuterium exchange mass spectrometry (HDX-MS), X-ray crystallography and cryo-electron microscopy (EM).

Methods of using the mAbs for antigen binding and evaluation of structural integrity and antigenicity, such as immunoassays (e.g., ELISA, Alphalisa, Luminex), and protein-protein interaction studies, such as by using Bio-Layer Interferometry (BLI) and Surface Plasmon Resonance (SPR).

### EMBODIMENTS

Isolated monoclonal antibodies that specifically bind HCMV Pentamer protein complex, and/or other gH-containing protein complexes, and methods of using such antibodies are provided herein. Antigen binding fragments of these antibodies, conjugates thereof, and methods of using the same are also provided. The antibodies of the present invention can be chimeric or humanized.

In one embodiment of the invention, isolated monoclonal antibodies (mAbs) and antigen binding fragments thereof include a heavy chain variable domain (VH) and a light chain variable domain (VL), wherein the heavy chain variable domain includes a heavy chain complementarity determining region (HCDR)1, an HCDR2 and an HCDR3, and wherein the light chain variable domain includes a light chain complementarity determining region (LCDR)1, an LCDR2 and an LCDR3, and wherein the mAb specifically binds to the pentamer protein complex (gH/gL/UL128/UL130/UL131A) of HCMV.

In a certain embodiment, the isolated mAb (or antigen binding fragment thereof) includes a heavy chain variable domain including the HCDR1, HCDR2, and HCDR3 sequences from SEQ ID NO: 1 as determined using a method selected from that of Paratome, Kabat, Chothia or IMGT, and a light chain variable domain including the LCDR1, LCDR2, and LCDR3 sequences from SEQ ID NO:2 as determined using a method selected from that of Paratome, Kabat, Chothia or IMGT, and wherein the HCDR1, HCDR2, HCDR3, LCDR1, LCDR2, and LCDR3 sequences are determined using the same method.

In a certain embodiment, the isolated mAb (or antigen binding fragment thereof) includes a heavy chain variable domain including the HCDR1, HCDR2, and HCDR3 sequences from SEQ ID NO:3 as determined using a method selected from that of Paratome, Kabat, Chothia or IMGT, and a light chain variable domain including the LCDR1, LCDR2, and LCDR3 sequences from SEQ ID NO:4 as determined using a method selected from that of Paratome, Kabat, Chothia or IMGT, and wherein the HCDR1, HCDR2, HCDR3, LCDR1, LCDR2, and LCDR3 sequences are determined using the same method.

In a certain embodiment, the isolated mAb (or antigen binding fragment thereof) includes a heavy chain variable domain including the HCDR1, HCDR2, and HCDR3 sequences from SEQ ID NO:5 as determined using a method selected from that of Paratome, Kabat, Chothia or IMGT, and a light chain variable domain including the LCDR1, LCDR2, and LCDR3 sequences from SEQ ID NO:6 as determined using a method selected from that of Paratome, Kabat, Chothia or IMGT, and wherein the HCDR1, HCDR2, HCDR3, LCDR1, LCDR2, and LCDR3 sequences are determined using the same method.

Recombinant nucleic acid molecules comprising one or more sequences encoding these heavy and light chain variable domains (SEQ ID NOs: 1-6) or CDRs (SEQ ID NOs: 7-24), expression vectors comprising such nucleic acid molecules, and host cells comprising such expression vectors are further embodiments of the invention.

In one embodiment of the isolated mAb or antigen binding fragment thereof, the HCDR1 comprises the amino acid sequence of SEQ ID NO: 7, the HCDR2 comprises the amino acid sequence of SEQ ID NO:8, the HCDR3 comprises the amino acid sequence of SEQ ID NO:9, the LCDR1 comprises the amino acid sequence of SEQ ID NO: 10, the LCDR2 comprises the amino acid sequence of SEQ ID NO: 11, and the LCDR3 comprises the amino acid sequence of SEQ ID NO: 12.

In one embodiment of the isolated mAb or antigen binding fragment thereof, the HCDR1 consists of the amino acid sequence of SEQ ID NO: 7, the HCDR2 consists of the amino acid sequence of SEQ ID NO: 8, the HCDR3 consists of the amino acid sequence of SEQ ID NO:9, the LCDR1 consists of the amino acid sequence of SEQ ID NO:10, the LCDR2 consists of the amino acid sequence of SEQ ID NO: 11, and the LCDR3 consists of the amino acid sequence of SEQ ID NO: 12.

In one embodiment of the isolated mAb or antigen binding fragment thereof, the HCDR1 comprises the amino acid sequence of SEQ ID NO: 13, the HCDR2 comprises the amino acid sequence of SEQ ID NO: 14, the HCDR3 comprises the amino acid sequence of SEQ ID NO:15, the LCDR1 comprises the amino acid sequence of SEQ ID NO: 16, the LCDR2 comprises the amino acid sequence of SEQ ID NO: 17, and the LCDR3 comprises the amino acid sequence of SEQ ID NO: 18.

In one embodiment of the isolated mAb or antigen binding fragment thereof, the HCDR1 consists of the amino acid sequence of SEQ ID NO: 13, the HCDR2 consists of the amino acid sequence of SEQ ID NO: 14, the HCDR3 consists of the amino acid sequence of SEQ ID NO: 15, the LCDR1 consists of the amino acid sequence of SEQ ID NO: 16, the LCDR2 consists of the amino acid sequence of SEQ ID NO: 17, and the LCDR3 consists of the amino acid sequence of SEQ ID NO:18.

In one embodiment of the isolated mAb or antigen binding fragment thereof, the HCDR1 comprises the amino acid sequence of SEQ ID NO: 19, the HCDR2 comprises the amino acid sequence of SEQ ID NO:20, the HCDR3 comprises the amino acid sequence of SEQ ID NO:21, the LCDR1 comprises the amino acid sequence of SEQ ID NO:22, the LCDR2 comprises the amino acid sequence of SEQ ID NO:23, and the LCDR3 comprises the amino acid sequence of SEQ ID NO:24.

In one embodiment of the isolated mAb or antigen binding fragment thereof, the HCDR1 consists of the amino acid sequence of SEQ ID NO: 19, the HCDR2 consists of the amino acid sequence of SEQ ID NO:20, the HCDR3 consists of the amino acid sequence of SEQ ID NO:21, the LCDR1 consists of the amino acid sequence of SEQ ID NO:22, the LCDR2 consists of the amino acid sequence of SEQ ID NO:23, and the LCDR3 consists of the amino acid sequence of SEQ ID NO:24.

It will be understood by those of skill in the art, that the similarity between two polypeptide sequences or two polynucleotide sequences can be expressed in terms of the similarity between the sequences, otherwise referred to as sequence identity. Sequence identity is frequently measured in terms of percentage identity (or similarity); the higher the percentage, the more similar are the primary structures of the two sequences. In general, the more similar the primary structures of two amino acid (or polynucleotide) sequences, the more similar are the higher order structures resulting from folding and assembly. Methods of determining sequence identity are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Sequence identity between polypeptide sequences is preferably determined by pairwise alignment algorithm using the Needleman-Wunsch global alignment algorithm (Needleman and Wunsch, A General Method Applicable to the Search for Similarities in the Amino Acid Sequence of Two Proteins, 1970 J. Mol. Biol. 48(3): 443-453), using default parameters (e.g. with Gap opening penalty = 10.0, and with Gap extension penalty = 0.5, using the EBLOSUM62 scoring matrix). This algorithm is conveniently implemented in the needle tool in the EMBOSS package (Rice et al., EMBOSS: The European Molecular Biology Open Software Suite, 2000 Trends Genetics 16: 276-277). Sequence identity should be calculated over the entire length of the identified reference sequence.

In one embodiment, the isolated monoclonal antibody or antigen binding fragment thereof comprises a heavy chain variable domain having an amino acid sequence at least 95%, 96%, 97%, 98%, 99% or 99.5% identical to SEQ ID NO:1. In a further embodiment, the isolated mAb or antigen binding fragment thereof comprises a light chain variable domain having an amino acid sequence at least 95%, 96%, 97%, 98%, 99% or 99.5% identical to SEQ ID NO:2. In a further embodiment, the isolated mAb or antigen binding fragment thereof includes both a heavy chain variable domain having an amino acid sequence at least 95%, 96%, 97%, 98%, 99% or 99.5% identical to SEQ ID NO:1, and a light chain variable domain having an amino acid sequence at least 95%, 96%, 97%, 98%, 99% or 99.5% identical to SEQ ID NO:2. In some embodiments, these isolated mAbs or antigen binding fragments thereof comprise the HCDRs of SEQ ID NOs: 7, 8, and 9. In some embodiments, these isolated mAbs or antigen binding fragments thereof comprise the LCDRs of SEQ ID NOs: 10, 11, and 12. In a further embodiment, the isolated mAb or antigen binding fragment thereof comprises both the HCDRs of SEQ ID NOs: 7, 8, and 9, and the LCDRs of SEQ ID NOs: 10, 11, and 12. In certain embodiments, these isolated mAbs (or antigen binding fragment thereof) specifically bind to HCMV Pentamer with an affinity (KD) of about 1.0 x 10-7 M or less, about 5.0 x 10-7 M or less, about 1.0 x 10-8 M or less, or about 5.0 x 10-8 M or less.

In one embodiment, the isolated mAb or antigen binding fragment thereof comprises a heavy chain variable domain having an amino acid sequence at least 95%, 96%, 97%, 98%, 99% or 99.5% identical to SEQ ID NO:3. In a further embodiment, the isolated mAb or antigen binding fragment thereof comprises a light chain variable domain having an amino acid sequence at least 95%, 96%, 97%, 98%, 99% or 99.5% identical to SEQ ID NO:4. In a further embodiment, the isolated mAb or antigen binding fragment thereof includes both a heavy chain variable domain having an amino acid sequence at least 95%, 96%, 97%, 98%, 99% or 99.5% identical to SEQ ID NO:3, and a light chain variable domain having an amino acid sequence at least 95%, 96%, 97%, 98%, 99% or 99.5% identical to SEQ ID NO:4. In some embodiments, these isolated mAbs or antigen binding fragments thereof comprise the HCDRs of SEQ ID NOs: 13, 14, and 15. In some embodiments, these isolated mAbs or antigen binding fragments thereof comprise the LCDRs of SEQ ID NOs: 16, 17, and 18. In a further embodiment, the isolated mAb or antigen binding fragment thereof comprises both the HCDRs of SEQ ID NOs: 13, 14, and 15, and the LCDRs of SEQ ID NOs: 16, 17, and 18. In certain embodiments, these isolated mAbs (or antigen binding fragment thereof) specifically bind to HCMV Pentamer with an affinity (KD) of about 1.0 x 10-7 M or less, about 5.0 x 10-7 M or less, about 1.0 x 10-8 M or less, or about 5.0 x 10-8 M or less.

In one embodiment, the isolated mAb or antigen binding fragment thereof comprises a heavy chain variable domain having an amino acid sequence at least 97%, 98%, 99% or 99.5% identical to SEQ ID NO:5. In a further embodiment, the isolated mAb or antigen binding fragment thereof comprises a light chain variable domain having an amino acid sequence at least 95%, 96%, 97%, 98%, 99% or 99.5% identical to SEQ ID NO:6. In a further embodiment, the isolated mAb or antigen binding fragment thereof includes both a heavy chain variable domain having an amino acid sequence at least 97%, 98%, 99% or 99.5% identical to SEQ ID NO:5, and a light chain variable domain having an amino acid sequence at least 95%, 96%, 97%, 98%, 99% or 99.5% identical to SEQ ID NO:6. In some embodiments, these isolated mAbs or antigen binding fragments thereof comprise the HCDRs of SEQ ID NOs: 19, 20, and 21. In some embodiments, these isolated mAbs or antigen binding fragments thereof comprise the LCDRs of SEQ ID NOs: 22, 23, and 24. In a further embodiment, the isolated mAb or antigen binding fragment thereof comprises both the HCDRs of SEQ ID NOs: 19, 20 and 21, and the LCDRs of SEQ ID NOs: 22, 23, and 24. In certain embodiments, these isolated mAbs (or antigen binding fragment thereof) specifically bind to HCMV Pentamer or to another gH-containing protein complex with an affinity (KD) of about 1.0 x 10-7 M or less, about 5.0 x 10-7 M or less, about 1.0 x 10-8 M or less, or about 5.0 x 10-8 M or less.

In a further embodiment, the isolated mAb or antigen binding fragment thereof comprises a heavy chain variable domain comprising or consisting of SEQ ID NO: 1. In a further embodiment, the isolated mAb or antigen binding fragment thereof comprises a light chain variable domain comprising or consisting of SEQ ID NO:2. In a further embodiment, the isolated mAb or antigen binding fragment thereof comprises a heavy chain variable domain of SEQ ID NO: 1 and a light chain variable domain comprising SEQ ID NO:2. In a further embodiment the isolated mAb or antigen binding fragment thereof comprises a heavy chain variable domain consisting of SEQ ID NO: 1 and comprises a light chain variable domain consisting of SEQ ID NO:2. In certain embodiments, these isolated mAbs (or antigen binding fragment thereof) specifically bind to HCMV Pentamer with an affinity (KD) of about 1.0 x 10-7 M or less, about 5.0 x 10-7 M or less, about 1.0 x 10-8 M or less, or about 5.0 x 10-8 M or less.

In a further embodiment, the isolated mAb or antigen binding fragment thereof comprises a heavy chain variable domain comprising or consisting of SEQ ID NO:3. In a further embodiment, the isolated mAb or antigen binding fragment thereof comprises a light chain variable domain comprising or consisting of SEQ ID NO:4. In a further embodiment, the isolated mAb or antigen binding fragment thereof comprises a heavy chain variable domain of SEQ ID NO:3 and a light chain variable domain comprising SEQ ID NO:4. In a further embodiment the isolated mAb or antigen binding fragment thereof comprises a heavy chain variable domain consisting of SEQ ID NO:3 and comprises a light chain variable domain consisting of SEQ ID NO:4. In certain embodiments, these isolated mAbs (or antigen binding fragment thereof) specifically bind to HCMV Pentamer with an affinity (KD) of about 1.0 x 10-7 M or less, about 5.0 x 10-7 M or less, about 1.0 x 10-8 M or less, or about 5.0 x 10-8 M or less.

In a further embodiment, the isolated mAb or antigen binding fragment thereof comprises a heavy chain variable domain comprising or consisting of SEQ ID NO:5. In a further embodiment, the isolated mAb or antigen binding fragment thereof comprises a light chain variable domain comprising or consisting of SEQ ID NO:6. In a further embodiment, the isolated mAb or antigen binding fragment thereof comprises a heavy chain variable domain of SEQ ID NO:5 and a light chain variable domain comprising SEQ ID NO:6. In a further embodiment the isolated mAb or antigen binding fragment thereof comprises a heavy chain variable domain consisting of SEQ ID NO:5 and comprises a light chain variable domain consisting of SEQ ID NO:6. In certain embodiments, these isolated mAbs (or antigen binding fragment thereof) specifically bind to HCMV Pentamer or to another gH-containing protein complex with an affinity (KD) of about 1.0 x 10-7 M or less, about 5.0 x 10-7 M or less, about 1.0 x 10-8 M or less, or about 5.0 x 10-8 M or less.

An embodiment of the present invention is antigen binding fragments, such as Fab, F(ab′)2, and Fv which include a heavy chain and VL and specifically bind HCMV Pentamer or another gH-containing protein complex. An antigen binding fragment of the present invention can comprise the heavy and light chain variable domains from the 26B6 mAb, the 23C2 mAb, or the 2C7 mAb as provided herein. Methods of making antigen binding fragments are known in the art, see e.g., Harlow and Lane, Antibodies: A Laboratory Manual, 2nd ed., Cold Spring Harbor Laboratory Press, New York, 2013.

The mAbs of the present invention can be mouse monoclonal antibodies, or can be humanized or chimeric antibodies, or antigen binding fragments thereof. The framework region can be any suitable framework region, such as (but not limited to) human, primate, monkey, rat, goat, sheep, rabbit, or mouse framework region. Human framework regions and amino acid mutations thereof are known in the art.

The isolated antibodies and antigen binding fragments of the present invention can be derivatized or linked to (conjugated to) another molecule, where derivatization does not significantly adversely affect specific binding to HCMV Pentamer, or to another gH-containing protein complex. Conjugation may use any suitable means as known in the art, and may be through either a covalent or non-covalent bond. A peptide linker (short peptide sequence) can be included between the two moieties. For example, the antibody or antigen binding fragment can be functionally linked to one or more molecules using chemical coupling, genetic fusion, or noncovalent association.

The isolated antibodies and antigen binding fragments of the present invention can be conjugated to a non-antigen molecule, such as a toxin, ligand, radioactive agent, or a detectable marker or label.

Detectable markers include those capable of detection by ELISA, spectrophotometry, flow cytometry, microscopy or other diagnostic imaging methods. Detectable markers include avidin/biotin, fluorophores, luminescent agents, radioactive isotopes and enzymatic linkages.

The isolated antibodies and antigen binding fragments of the present invention can be modified using methods known in the art to increase in vivo circulating half-life (compared to an unmodified molecule), e.g., increase the circulating half-life when administered to a subject for prophylactic or therapeutic purposes. Such modifications include amino acid substitutions in the Fc region (see e.g., U.S. Pat. Application No. 2020/0071423, Tsui et al.; Dall’Acqua et al. (2002) J. Immunol. 169, 5171-5180; Dall’Acqua et al. (2006) J. Biol. Chem. 281, 23514-23524; Datta-Mannan et al. (2007) Drug Metab. Dispos. 35, 86-94; Deng et al. (2010) Drug Metab. Dispos. 38, 600-605; Hinton et al. (2004) J. Biol. Chem. 279, 6213-6216; Hinton et al. (2006) J. Immunol. 176, 346-356; Yeung et al. (2009) J. Immunol. 182, 7663-7671); Zalevsky et al. (2010) Nat. Biotechnol. 28, 157-159). IgGs having extended in vivo half-lives as a result of modification of an IgG constant domain are also described in U.S. Pat Nos. 7,083,784, 7670,600, 7,704,497, 8,012,476, 8,323,962, 8,475,792 and WO2002/060919 (Dall’Acqua et al.). Amino acid changes may readily be made in the antibodies and antigen binding fragments of the invention, and screened using well-known and routine assays to identify those resulting in increased in vivo half-life.

Additional methods of extending in vivo circulating half-life include pI (isoelectric point) engineering, PEGylation, and Pasylation. The term “in vivo half-life” as used herein means a pharmacokinetic property of a molecule that is a measure of the mean survival time of the molecules following their administration. Half-life can be expressed as the time required to eliminate fifty percent (50%) of a known quantity of the molecule from a subject’s body (e.g., a human patient or other mammal) or a specific compartment thereof, for example, as measured in serum (i.e., “in vivo circulating half-life”), or in other tissues. In general, an increase in half-life results in an increase in mean residence time (MRT) in circulation for the molecule administered.

### Assay Kits and Devices

Antibodies of the present invention can be included in a kit, container, pack, or dispenser together with instructions for their utilization.

The invention further provides an assay kit and an assay device comprising an isolated antibody or antigen binding fragment of the present invention. Assay kits (which may also be referred to as reagent kits) comprise an isolated antibody or an isolated binding fragment of the invention, which may be coupled to a labeling or detectable moiety. The kit may also one or more calibrators comprising a known amount of an HCMV Pentamer (or other gH-containing complex).

The phrase “assay kit” refers to an assembly of materials and reagents that is used in performing an assay. The reagents can be provided in packaged combination in the same or in separate containers, depending on their cross-reactivities and stabilities, and in liquid or in lyophilized form. The amounts and proportions of reagents provided in the kit can be selected to provide optimum results for a particular application. An assay kit embodying features of the present invention comprises antibodies or binding fragments thereof which bind HCMV Pentamer or other gH-containing complexes. The kit may further comprise competitive binding partners of the HCMV proteins, calibration and control materials.

The phrase “calibration and control material” refers to any standard or reference material containing a known amount of an analyte. A sample suspected of containing an analyte and the corresponding calibration material are assayed under similar conditions. The concentration of analyte is calculated by comparing the results obtained for the unknown specimen with the results obtained for the standard. This is commonly done by constructing a calibration curve.

Isolated antibodies or isolated binding fragments of the present invention may also be provided as part of an assay device. Such assay devices include lateral flow assay devices, such as those commonly known as lateral flow test strips. Another type of assay device is a non-porous assay device having projections to induce capillary flow.

### Nucleotide Molecules, Vectors and Host Cells

Nucleic acid molecules encoding the polypeptides of the present invention (e.g., antibodies, antigen binding fragments, VH, VL, HCDRs, and LCDRs), expression vectors comprising such nucleic acid molecules, and host cells comprising such vectors are also provided. As will be apparent to those skilled in the art, such nucleic acid molecules, expression vectors and host cells can be used in methods of producing the polypeptides of the invention.

Nucleic acid molecules encoding the polypeptides of the present invention may also be administered to a subject, in prophylactic or therapeutic methods. Such methods may be referred to as DNA or RNA vaccination and result in expression of the encoded molecule in the subject. Thus, an embodiment of the present invention is compositions comprising such nucleic acid molecules, pharmaceutical formulations comprising such compositions, and methods of treatment or prophylaxis of HCMV infection or disease, comprising administering such compositions or formulations to a subject in need thereof. The nucleic acid molecules may be of any suitable form, including plasmid DNA and mRNA.

Nucleic acid sequences encoding antibodies and antigen binding fragments of the present invention can be readily determined and produced by one of ordinary skill in the art, using methods as known in the art, and based on the polypeptide sequences provided herein, or a combination of the polypeptide sequences provided herein (such as CDR sequences) and polypeptide sequences available in the art (such as framework or constant region sequences). The nucleic acid sequences of the invention can also encode a polypeptide of the present invention that contains additional amino acids that aid in purification of the expressed polypeptide, such as a C-terminal tail of ten or fewer amino acids, such as histidine and/or glycine residues.

It will be apparent to one of skill in the art that, due to the degeneracy of the genetic code, more than one nucleic acid sequence can encode the same polypeptide. Nucleic acid sequences of the present invention may be codon optimized, as is known in the art, to improve expression in a selected host cell. Nucleic acid sequences of the present invention can be prepared by any suitable method as is known in the art, including by recombinant methods, amplification (e.g., polymerase chain reaction), or chemical synthesis.

Nucleic acid molecules encoding polypeptides of the present invention can be operably linked to other nucleic acid sequences that regulate expression (such as promoters, enhancers, transcription and translation terminators, initiation sequences, ribosome binding sites, start codons, and stop codons). Expression vectors comprising such nucleic acid molecules can be expressed in a host cell to provide the encoded polypeptide. Expression vectors can be transferred into the selected host cell by any suitable method as is known in the art, such as microinjection, transformation, electroporation, or lipofection, or the use of viral vectors.

Nucleic acid sequences of the present invention can be expressed in recombinantly engineered host cells (including bacterial, insect, plant, yeast, insect and mammalian cells), and the expressed polypeptide purified (see, e.g., Al-Rubeai (ed.), Antibody Expression and Production, Springer Press, 2011; Harlow and Lane, Antibodies: A Laboratory Manual, 2nd ed., Cold Spring Harbor Laboratory Press, New York 2013).

A further embodiment of the present invention is a host cell comprising a recombinant expression vector as described herein, or a population of such host cells. Expressed polypeptides can be purified according to methods as are known in the art, such as by column chromatography, affinity columns, or ammonium sulfate precipitation. See, e.g., Simpson (ed.) Basic Methods in Protein Purification and Analysis: A Laboratory Manual, Cold Spring Harbor Laboratory Press, 2008.

### Compositions

A further embodiment of the present invention is a composition comprising one or more of the isolated antibodies, antigen binding fragments, or conjugates of the present invention, in a carrier. The carrier can be a pharmaceutically acceptable carrier, as is known in the art. See, e.g., Remington’s Pharmaceutical Science, 22nd ed., Pharmaceutical Press, London, UK (2012). Pharmaceutically acceptable carriers include fluids such as sterile water for injection, physiological saline, and balanced salt solutions. Pharmaceutical compositions of the present invention can be sterile and can include additional substances such as wetting or emulsifying agents, preservatives, and/or pH buffering agents.

Isolated antibodies, antigen binding fragments, and conjugates of the present invention can be lyophilized, and subsequently rehydrated with sterile water prior to use. Accordingly, one embodiment of the present invention is a lyophilized antibody, antigen binding fragment, or conjugate as described herein, or a composition comprising such lyophilized molecules.

### Methods of Detection

The isolated antibodies and antigen binding fragments of the present invention can be used in methods of detecting the presence of HCMV Pentamer (or other gH-containing complex) in a composition or sample, such as a biological sample obtained from a subject. Such methods include contacting the composition being tested, or a portion thereof (a ‘test sample’), with an isolated antibody or antigen binding fragment of the present invention, under conditions that allow an immune complex to be formed between any HCMV Pentamer (or gH-containing complex) present in the sample and the antibody or antigen binding fragment. Any immune complex formed is then detected and indicates that HCMV Pentamer (or gH-containing complex) was present in the sample. Absence of detectable immune complex is interpreted as indicating the absence of the HCMV Pentamer (or gH-containing complex) and may be referred to as ‘detecting the absence.’

Additionally, the amount of immune complex formed may be used to assess the concentration of HCMV Pentamer (or gH-containing complex) present in the sample. Any suitable immunoassay as is known in the art may be used to detect the presence of the immune complex, including but not limited to competitive and non-competitive assays using techniques such as Western blots, Enzyme Linked Immunosorbant Assay (ELISA), immunoprecipitation, radioimmunoassay, immunodiffusion assays, fluorescent immunoassays, lateral flow assays (LFA) and microfluidic assays.

The isolated antibodies and antibody binding fragments of the invention can be used to assess the structural integrity of the HCMV Pentameric complex; i.e., to determine whether the complex is folded in such a way to correctly present the neutralizing epitope bound by the mAb or binding fragment utilized in the assay.

Certain isolated antibodies and antigen binding fragments of the present invention can be used in methods of detecting the presence of HCMV gH/gL/gO protein complex in a composition or sample, such as a biological sample obtained from a subject. Such methods include contacting the composition being tested, or a portion thereof (a ‘test sample’), with an antibody or antigen binding fragment of the present invention, under conditions that allow an immune complex to be formed between any HCMV gH/gL/gO present in the sample and the antibody or antigen binding fragment. Any immune complex formed is then detected, and indicates that HCMV gH/gL/gO was present in the sample. Absence of detectable immune complex is interpreted as indicating the absence of the HCMV gH/gL/gO, and may be referred to as ‘detecting the absence.’

Additionally, the amount of immune complex formed may be used to assess the concentration of HCMV gH/gL/gO present in the sample. Any suitable immunoassay as is known in the art may be used to detect the presence of the immune complex, including but not limited to competitive and non-competitive assays using techniques such as Western blots, Enzyme Linked Immunosorbant Assay (ELISA), immunoprecipitation, radioimmunoassay, immunodiffusion assays, fluorescent immunoassays, lateral flow assays (LFA) and microfluidic assays.

Certain isolated antibodies and antibody binding fragments of the invention can be used to assess the structural integrity of the gH/gL/gO complex; i.e., to determine whether the complex is folded in such a way to correctly present the neutralizing epitope bound by the mAb or binding fragment utilized in the assay.

Certain isolated antibodies and antibody binding fragments of the invention can be used to assess the structural integrity of HCMV gH/gL/gO complex; i.e., to determine whether the complex is folded in such a way to correctly present the neutralizing epitope bound by the mAb or binding fragment utilized in the assay.

In certain embodiments of the present invention, the isolated antibody or antigen binding fragment used in a method of detection is conjugated to a detectable marker. Alternative methods utilize a second antibody that specifically binds to the antibody, antigen binding fragment, or conjugate thereof used in the method, to form an immune complex that is then detected. The second antibody can be conjugated to a detectable marker.

A ‘biological sample’ obtained from a subject includes cells and tissues, as well as bodily fluids such as blood, fractions of blood (e.g. serum or plasma), cerebrospinal fluid, urine, and sputum.

### Terms

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “~”) 200 pg.

As used herein, “Pentamer” refers to the HCMV protein complex pentamer of gH, gL, UL128, UL130, and UL131A, which may also be referred to as “HCMV Pentamer,” “gH/gL/UL128/UL130/UL131A,” or “gH/gL/UL128-131”.

As used herein, “administration” refers to the introduction of a compound or composition into a subject by a chosen route as is known in the art. Exemplary routes of administration include oral, intravenous, subcutaneous, intramuscular, intradermal, intraperitoneal, sublingual, rectal, transdermal, intranasal, vaginal and inhalation.

As used herein, “antibody” refers to an immunoglobulin that specifically binds to an analyte (such as a protein or protein complex, such as HCMV Pentamer or other gH-containing complexes). Non-limiting examples of antibodies include intact immunoglobulins as well as variants and/or fragments thereof that retain the binding affinity of the intact immunoglobulin for the antigen.

As used herein, and “antigen binding fragment” of an antibody includes Fv, Fab, Fab′, Fab′-SH, F(ab′)2 constructs; diabodies; linear antibodies; single-chain antibody molecules (e.g.,scFv); and multispecific antibodies formed from antibody fragments. Antibody fragments include antigen binding fragments whether produced by the modification of whole antibodies or those synthesized de novo using chemical synthesis or recombinant DNA methodologies (see, e.g., Konermann and Dubel (Ed.), Antibody Engineering, Vols. 1-2, 2nd Ed. Springer Press, 2010). Single-chain antibodies (scFv) contain the VH and VL domains of one or more antibodies linked by a suitable polypeptide linker as a fused single chain molecule; scFv antibodies may be recombinantly engineered. Peptide sequences are conventionally written N-terminus to C-terminus, left to right, in languages read left-to-right. In scFv antibodies, either the VH-domain or the VL-domain may be at the N-terminus (VH-linker-VL, or VL-linker-VH). In disulfide-stabilized Fv (dsFv) fragments, the VH and VL are stabilized by an interchain disulfide bond.

As used herein, a polypeptide having a sequence “from” a larger sequence is a polypeptide that has a contiguous linear sequence of amino acids identical to a contiguous linear sequence of amino acids found in the larger sequence; in other words, the polypeptide having a sequence “from” a larger sequence is a subsequence of the larger sequence. Such a polypeptide may be sequenced de novo; it need not be physically obtained by fragmenting a larger sequence. This applies to HCDR and LCDR sequences described as “of” or “from” an identified longer sequence.

As used herein, ‘antibody’ includes genetically engineered (recombinant) antibody forms such as chimeric antibodies (e.g., humanized murine antibodies) and heteroconjugate antibodies (such as bispecific antibodies).

As used herein, an ‘epitope’ refers to an antigenic determinant, the particular peptide sequences or chemical groups on a molecule that are antigenic (elicit a specific immune response). An antibody specifically binds a particular antigenic epitope. Isolated antibodies of the present invention bind to particular epitopes on HCMV Pentamer, gH/gL, or gH/gL/gO complexes.

An antibody that “binds to the same epitope” as a reference antibody refers to an antibody that blocks specific binding of the reference antibody to its antigen (the target antigen) in a competition assay by at least 50%. Conversely, the reference antibody blocks specific binding of the tested antibody to the target antigen in a competition assay by at least 50%. Any suitable antibody competition assay as is known in the art may be used in assessing the antibodies of the present invention.

Typically, a naturally occurring immunoglobulin has heavy chains and light chains interconnected by disulfide bonds. Immunoglobulin genes include the kappa, lambda, alpha, gamma, delta, epsilon and mu constant region genes, as well as immunoglobulin variable domain genes. There are two light chain types (lambda and kappa), and five main heavy chain classes (or isotypes) (IgM, IgD, IgG, IgA and IgE). Each light and heavy chain contains a constant region and a variable domain. “VH” or “VH” refers to an antibody heavy chain variable domain. “VL” or “VL” refers to an antibody light chain variable domain. VH and VL variable domains contain a ‘framework’ region. Interspersed within each framework region are three hypervariable domains (also referred to as complementarity-determining regions or CDRs) (see e.g., Kabat et al., Sequences of Proteins of Immunological Interest, 5th Ed. Public Health Service, National Institutes of Health, Bethesda, MD, 1991). The sequences of the framework regions of different light or heavy chains are relatively conserved within a species. The framework regions function to position the CDRs in three-dimensional space. Heavy chain CDRs are typically referred to as HCDR1, HCDR2 and HCDR3 (from N- to C-terminus). Light chain CDRs are typically referred to as LCDR1, LCDR2 and LCDR3 (from N- to C-terminus). As used herein, reference to an HCDR or LCDR encompasses the corresponding Paratome ABR.

As used herein, a ‘monoclonal antibody’ (mAb) is an antibody obtained from a population of substantially identical antibodies, i.e., the antibodies in the population bind the same epitope. It is however recognized that a population of mAbs may contain a minority of variant antibodies (e.g., antibodies containing naturally-occurring mutations such as those arising during production of mAbs). In contrast, polyclonal antibody preparations typically include different antibodies directed against different epitopes. Monoclonal antibodies of the present invention may be produced or obtained by any method as is known in the art, including hybridoma methods, recombinant DNA production, phage-display methods, and use of transgenic animals containing all or part of the human immunoglobulin loci. Monoclonal antibodies of the present invention may contain conservative amino acid substitutions, where such substitutions have substantially no effect on the antibody function.

As used herein, a ‘humanized’ antibody or antigen binding fragment includes a human framework region and one or more CDRs from a non-human antibody or antigen-binding fragment. Such non-human antibody or antigen-binding fragment sources include mouse, rat, or synthetic sources. The non-human antibody or antigen-binding fragment that provides the CDRs is referred to as the ‘donor’, and the human framework is termed the ‘acceptor.’ In one embodiment, all the CDRs of a humanized antigen or antigen-binding fragment are from a single donor antigen or antigen-binding fragment.

As used herein, a ‘chimeric’ antibody includes sequences derived from two different antibodies, which may originate from different species.

As used herein, a “recombinant” or “engineered” cell refers to a cell into which an exogenous DNA sequence, such as a cDNA sequence, has been introduced. A “host cell” is one that contains such an exogenous DNA sequence.

“Recombinant” as used herein to describe a polynucleotide means a polynucleotide which, by virtue of its origin or manipulation: (1) is not associated with all or a portion of the polynucleotide with which it is associated in nature; (2) is linked to a polynucleotide other than that to which it is linked in nature; and/or (3) has a sequence that is not naturally occurring. The term “recombinant” as used with respect to a protein or polypeptide means a polypeptide produced by expression of a recombinant polynucleotide.

As used herein, a ‘biological sample’ is a sample obtained from a subject, including samples obtained for detection of disease or infection. Biological samples include cells, tissues, and bodily fluids (e.g., blood, urine, and cerebrospinal fluid).

As used herein, an ‘immune complex’ refers to a complex created by the binding of an antibody (or antigen-binding fragment thereof) to a soluble antigen. Formation of an immune complex can be detected using methods as are known in the art. Immunological binding properties of a selected antibody can be quantified using methods as are known in the art.

As used herein, ‘conditions sufficient to form an immune complex’ are conditions that allow an antibody (or antigen-binding fragment) to bind to its cognate epitope to a detectably greater degree than, and/or to the substantial exclusion of, binding to other epitopes. Such conditions depend upon the format of the binding reaction and may be determined using methods as are known in the art. See, e.g., Harlow and Lane, Antibodies: A Laboratory Manual, 2nd ed. Cold Spring Harbor Laboratory Press, New York (2013). Immune complexes can be detected through conventional methods as are known in the art, e.g., immunohistochemistry, immunoprecipitations, flow cytometry, immunofluorescence microscopy, ELISA, immunoblotting, and chromatography. Methods are also known in the art for quantifying the immunological binding properties of an antibody.

As used herein, a ‘conservative amino acid substitution’ is an amino acid substitution that does not substantially affect or decrease s function of a protein, such as the ability of an antibody to specifically bind to an antigen. Conservative amino acid substitution tables that provide functionally similar amino acids are readily available and known to those skilled in the art. The following groups are examples of amino acids that are considered as conservative substitutes for each other:
(1) Alanine (A), Serine (S), Threonine (T);
(2) Aspartic acid (D), Glutamic acid (E);
(3) Asparagine (N), Glutamine (Q);
(4) arginine (R), Lysine (K);
(5) Isoleucine (I), Leucine (L), Methoionine (M), Valine (V); and
(6) Phenylalanine (F), Tyrosine (Y), Tryptophan (W).

As used herein, ‘non-conservative’ amino acid substitutions are those that reduce an activity or function of an antibody of the invention, such as the ability to specifically bind to HCMV Pentamer.

As used herein, ‘contacting’ refers to placing items or molecules in direct physical association, including in either solid or liquid form.

As used herein, a ‘control’ is a reference standard, and may be a negative control, a positive control, a historical control, or a standard reference value or range of values.

As used herein, a ‘detectable marker’ or ‘label’ refers to a molecule capable of detection by methods as are known in the art. Detectable markers can be conjugated (directly or indirectly) to a polypeptide of the present invention to facilitate detection. Examples of detectable markers include avidin, biotin, fluorophores, chemiluminescent agents, enzymatic linkages, radioactive isotopes, and heavy metals or compounds. See e.g., Sambrook et al., (Molecular Cloning: A Laboratory Manual, 4th ed., Cold Spring Harbor Laboratory Press, New York, 2012). A ‘detectably labeled antibody’ refers to the combination of antibody and detectable marker.

As used herein, ‘expression’ refers to transcription or translation of a nucleic acid sequence. An ‘expression vector’ is a vector comprising a recombinant polynucleotide sequence to be expressed and comprising expression control sequences operatively linked to the recombinant sequence to be expressed. Expression vectors are known in the art, and include cosmids, plasmids, and viruses.

As used herein, ‘isolated’ refers to a biological component (such as a nucleic acid molecule, a peptide or protein, an antibody or antigen binding fragment thereof) that has been substantially separated, produced separately from, or purified from other biological components in the cell in which the component naturally occurs. Isolated peptides and proteins include those purified by standard purification methods, e.g., proteins produced recombinantly in a cell and purified from the cell culture, or those chemically synthesized. ‘Purification’ does not imply 100% purity, i.e., the complete exclusion of all other components. An isolated nucleic acid molecule, peptide, or protein is at least 50%, at least 60%, at least 70%, at least 80%, at least 90%, at least 95%, at least 96%, at least 97%, at least 98%, or at least 99% pure.

As used herein, a ‘linker’ is a bifunctional molecule that can be used to link two molecules together, to create one contiguous molecule, for example to link a detectable marker to an antibody.

As used herein, a ‘nucleic acid molecule’ (or nucleic acid sequence) is a polymer of nucleotides, which may include both sense and anti-sense strands of RNA, cDNA, genomic DNA, and synthetic forms and mixed polymers of the above.

A ‘degenerate variant’ of a reference polynucleotide sequence refers to a variant polynucleotide sequence that encodes the same protein as the reference polynucleotide sequence, but where the nucleotide sequence differs due to the degeneracy of the genetic code. ‘Encodes’ or ‘encoding’ refers to the inherent property of specific sequences of nucleotides in a polynucleotide (such as a gene, a cDNA, or an mRNA) to act as a template for the synthesis of other polymers and/or macromolecules.

As used herein, ‘operably linked’ refers to the functional relationship of two nucleic acid sequences. For example, a promoter is operably linked to a coding sequence when the promoter affects or controls the transcription or expression of the coding sequence.

As used herein, a ‘polypeptide’ is a polymer of amino acid residues joined via amide bonds as produced by natural or recombinant means. The terms polypeptide, peptide, and protein are used interchangeably herein.

As used herein ‘specific binding’ of an antibody or antigen binding fragment refers to a binding reaction to a target protein. Under designated conditions, an antibody or antigen binding fragment binds preferentially to its target protein and does not bind in significant amounts to other proteins or molecules present in a sample being tested for the presence of the target protein. Specific binding conditions can be determined using methods as are known in the art. Specific binding of an antibody and an antigen can refer to a KD of less than about 10-7 Molar, or less than about 10-8 Molar, or less than about 10-9 Molar. ‘KD’ refers to the dissociation constant for a given interaction, such as a polypeptide ligand interaction or an antibody-antigen interaction.

As used herein, a “subject” is a living multi-cellular vertebrate organism, such as a non-human mammal, e.g., a mouse, a rat, or a non-human primate. Alternatively, the subject can be a human subject.

As used herein, ‘treatment’ using an antibody, antigen binding fragment, or composition of the present invention refers to both prophylactic use and therapeutic use against HCMV infection. Prophylactic use occurs prior to HCMV infection and results in a reduction, in a population of treated subjects, of the number of subjects that become infected (compared to an untreated control population). Stated another way, prophylactic treatment reduces the risk of developing HCMV infection, compared to the risk in an untreated control population. Therapeutic use is applicable to subjects infected with

HCMV, and results in amelioration of symptoms of HCMV infection (e.g., a reduction in the severity of symptoms or a reduction in the time the symptoms are present in a subject), compared to untreated control subjects.

As used herein, an ‘effective amount’ refers to the amount that induces a desired response, such as prevention or inhibition of viral infection, or reduction of symptoms caused by a viral infection. As used herein, a ‘therapeutically effective amount’ is an amount necessary to achieve therapeutic results; a ‘prophylactically effective amount’ is an amount necessary to achieve prophylaxis. Such amount will vary depending upon the subject and the subject’s health, and may be determined using methods as are known in the art. Both prophylactic and therapeutic treatment comprise administration of an effective amount of the antibody, antigen binding fragment, or composition of the present invention, to a subject in need of such treatment.

As used herein, a ‘transformed’ cell is a cell into which a nucleic acid molecule has been introduced using molecular biological techniques, such as transformation with viral or plasmid vectors, or the use of electroporation or lipofection.

As used herein, a ‘vector’ is a recombinant construct comprising nucleic acid sequences that enable replication in a host cell. A vector can comprise a selectable marker. Viral vectors comprise nucleic acid sequences derived from one or more viruses.

Suitable materials and methods for the practice of the present invention are described herein. Additional suitable methods and materials may be used as will be apparent to those skilled in the art. See e.g., Sambrook et al., Molecular Cloning: A Laboratory Manual, 4th ed. Cold Spring Harbor Laboratory Press, 2012; Sambrook et al., Molecular Cloning: A Laboratory Manual, 3rd ed. Cold Spring Harbor Laboratory Press, 2001; Ausubel et al., Current Protocols in Molecular Biology, Greene Publishing Associates, 1992 (and supplements to 2012); Ausubel et al., Short Protocols in Molecular Biology: A Compendium of Methods from Current Protocols in Molecular Biology, 4th Ed. Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is derived from the Latin exempli gratia and is used herein to indicate a non-limiting example. Thus, the abbreviation “e.g.” is synonymous with the term “for example.”

In order to facilitate review of the various embodiments of this disclosure, the following explanations of terms are provided. Additional terms and explanations can be provided in the context of this disclosure.

### EXAMPLES

### Example 1: Generation of Humanized mAbs From Pentamer Immunized Mice

Mice were immunized with soluble HCMV Pentamer. After immunizations, the spleens of the mice were harvested and fused with myeloma cells to generate multiple hybridoma cells, and hybridoma supernatants were screened for binding to HCMV Pentamer. Reactive hybridoma supernatants were then tested in HCMV neutralization assays on epithelial cells. The three hybridoma supernatants showing the highest neutralization titers were selected. The murine antibodies isolated from the three supernatants are referred to as m26B6, m23C2 and m2C7. The Variable Heavy and Variable Light chains of m26B6, m23C2 and m2C7 were sequenced (SEQ ID NOs: 1- 6).

Humanized mAbs were constructed by subcloning nucleic acid sequences encoding the heavy and light chain variable domains of each murine antibody into a mammalian expression plasmid containing human IgG1 backbone. Thus, three humanized mAbs were prepared: 26B6 (containing heavy and light variable chains of m26B6), 23C2 (containing heavy and light variable chains of m23C2), and 2C7 (containing heavy and light variable chains of m2C7). These humanized mAbs were used for functional and structural characterization as described below.

Humanized mAbs were constructed by subcloning nucleic acid sequences encoding the heavy and light chain variable domains of each murine antibody into a mammalian expression plasmid containing human IgGl backbone. Thus, three humanized mAbs were prepared: 26B6 (containing heavy and light variable chains of m26B6), 23C2 (containing heavy and light variable chains of m23C2), and 2C7 (containing heavy and light variable chains of m2C7). These humanized mAbs were used for functional and structural characterization as described below.

### Example 2: Epitope Binning Assay and Binding to Pentamer and gH/gL/gO

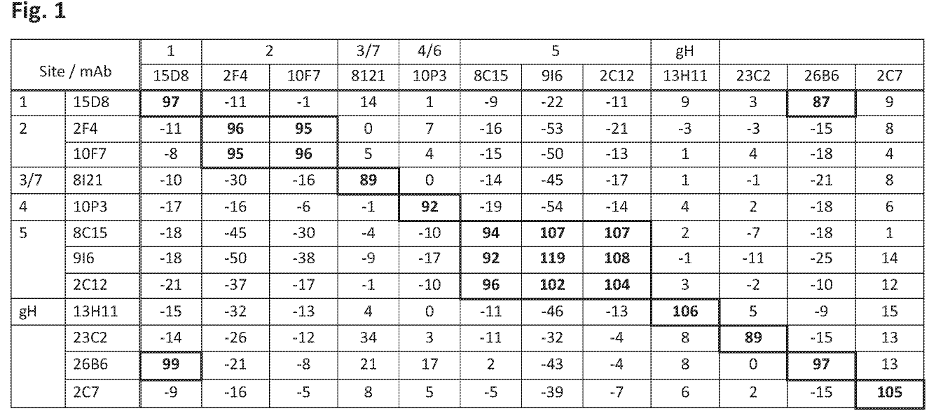

Antibody competition assay on HCMV Pentamer was performed using the Octet RED96 instrument (ForteBio Corp). Pairs of mAbs (as shown in FIG. 1) were tested for binding to Pentamer. Pentamer and mAbs were diluted in 1 x Phosphate Buffered Saline (PBS) with 1% Bovine Serum Albumin (BSA) at the final concentration of 20 µg/ml and the assay was run at 30° C.

The monoclonal antibody 3G16, isolated from immortalized memory B-cells of HCMV-immune donors, is known to bind to HCMV glycoprotein H (gH). See e.g., Macagno et al., J Virol 84: 1005 (2010); Ciferri et al., PLoS Pathog 11(10):e1005230 (2015).

Biotinylated 3G16 (Biot-3G16) was captured onto Streptavidin biosensors (ForteBio Corp) for 60 seconds 1× PBS with 1% BSA. Typical capture levels were between 0.5 and 0.7 nanometer (nm). Biosensor tips were washed for 30 seconds 1_{X} PBS with 1% BSA before capturing Pentamer for 30 seconds. An additional washing step was done before capturing primary and secondary Mabs. Binding of primary and secondary mAbs was measured for 300 seconds. Binding inhibition was calculated by the following equation: inhibition (%) = 100 - (secondary MAb binding in the presence of primary mAb) / (secondary MAb binding in absence of primary mAb) x 100. The values reported are the average of two independent experiments.

A panel of HCMV neutralizing antibodies (eight UL-specific antibodies and one gH-specific antibody) was tested in a competition assay to identify the epitopes bound by 26B6, 23C2 and 2C7 mAbs (FIG. 1).

Binding sites of antibodies listed in Table 7 were previously identified. The position of the binding sites on the Pentamer are described by Ciferri et al., PLoS Pathog 11(10):e1005230 (2015).

TABLE 7

Comparator Antibodies

| | |
|---|---|
| Monoclonal Antibody | Binds to epitope in |
| 15D8* | Site 1 (ULs) |
| 2F4* | Site 2 (ULs) |
| 10F7* | Site 2 (ULs) |
| 8\|21* | Site 3/7 (ULs) |
| 10P3* | Site 4 (ULs) |
| 8C15* | Site 5 (ULs) |
| 9\|6* | Site 5 (ULs) |
| 2C12* | Site 5 (ULs) |
| 4\|22* | Site 2 (Uls) |
| 3G16* | gH |
| 13H11* | gH |
| MSL-109⁺ | gH |

*human mAb isolated from immortalized memory B-cells of HCMV-immune donors; see Macagno et al., J Virol 84:1005-1013 (2010) pmid:19889756
+human mAb isolated from a CMV seropositive individual; see Fouts et al., Proc Natl Acad Sci USA 111(22):8209-14 (2014).

Results are shown in FIG. 1. Bolded cells indicate 100% competition for the same neutralizing epitopes.

26B6 was able to compete with mAb 15D8, indicating an overlapping epitope on Site 1; while 23C2 and 2C7 did not compete with any known neutralizing epitope, suggesting the presence of previously un-identified epitopes.

To further define epitopes bound by 23C2 and 2C7, binding to Pentamer and gH/gL/gO was tested using Surface Plasmon Resonance (SPR) binding analysis. Single-cycle kinetic experiments were carried out by using a Biacore 8 K+ instrument. All 8 channels on a Protein A sensor chip were used to carry out the experiment. HBS-EP buffer (GE Healthcare BR100669) was used as running buffer, and diluent for both ligand and analyte samples. Ligands 23C2, 26B6 and 2C7 IgG were captured on one channel (leaving the other channel as reference) to about 180-200 response units. Varying concentrations of Pentamer or gH/gL/gO ranging from 2.5 to 20 nM were injected over the two channels at 40 µL/min for 120 seconds, followed by 600 seconds of dissociation time. The single-cycle kinetic curves were fitted with 1:1 binding stoichiometry for kon, koff, and KD. Each sample was run in triplicate.

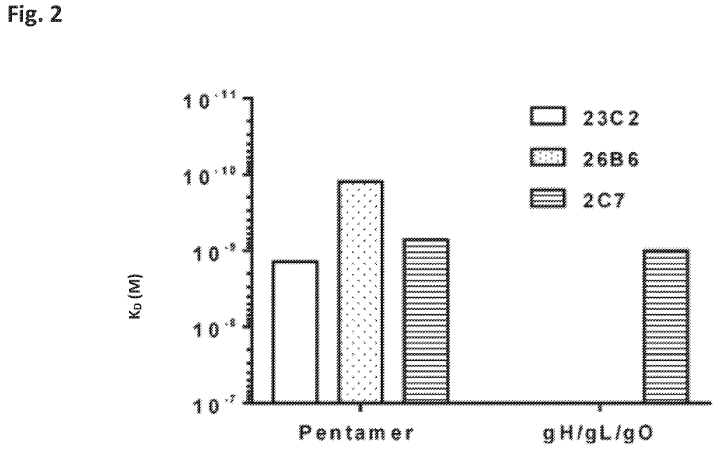

Results are graphed in FIG. 2. The graph bar indicates the affinity (KD) of each mAb for Pentamer and gH/gL/gO. Each bar represents the average of three experiments. 26B6 bound only to Pentamer, confirming the location of its epitope on Site 1. 23C2 was able to interact only with Pentamer, demonstrating that this mAb could bind an epitope on UL units (Table 8 And FIG. 2). 2C7 bound both Pentamer and gH/gL/gO, suggesting an epitope on the gH/gL complex (FIG. 2).

### Example 3: Neutralization Properties

HCMV uses different entry mechanisms for infection of epithelial cells versus fibroblasts. The Pentameric complex plays a crucial role in receptor recognition and subsequent entry into epithelial, endothelial and myeloid cells, while it is dispensable for entry into fibroblasts. The gH/gL/gO complex plays a critical role in HCMV entry into fibroblasts by binding to its cognate receptor, platelet derived growth factor receptor (PDGFR). To evaluate the neutralization properties of 26B6, 23C2 and 2C7, these mAbs were tested against HCMV in both epithelial cells (human retinal pigment cells, ARPE-19) and fibroblasts (human lung fibroblast cells, MRC-5). A set of seven previously characterized neutralizing antibodies were included as control (see Table 7 and Table 8).

HCMV neutralization assay method: cells were seeded at 8.5x104/ml (ARPE-19; ATCC) or 2.0x105/ml (MRC5; ATCC) in 96-well Edge 2.0 flat bottom plates (Thermo Scientific) and incubated overnight at 37° C. with 5% C02. The following day, antibody dilutions were performed in DMEM media (Gibco) containing 2% heat-inactivated FBS (Gibco) and 1% Penicillin/ streptomycin/L-Glutamine (Gibco) solution. Positive control sera, ACCURUN 145 CMV IgG (SeraCare) and 1G2 were also included on every plate. For virus dilution preparation, DMEM media was supplemented with or without guinea pig complement (Cederlane) and the solution was filtered prior to virus dilution. The volume of TB40 virus stock to use was previously determined by infectivity assay. Briefly, the amount of virus added per well was titrated to give 150-200 GFP positive cells in control wells.

For the neutralization assay 10-fold serial dilutions of each antibody were mixed with equal volumes (75 µl) of diluted virus, starting at 10 µg/ml. The virus-sera mixture was incubated for 2 hours at 37° C. with 5% C02.

Culture medium from cells was removed and 50 µl of the sera-virus mixture was transferred to cells. Each sample was tested in duplicate. After overnight incubation at 37° C. with 5% C02, cells were washed once with 1x PBS and fixed with 4% paraformaldehyde (ThermoFisher) for 20 minutes. Cells were washed once with 1x PBS and then permeabilized with 0.1% tritonX100 (EMD Millipore) for 10 minutes. After 10 minutes, cells were washed twice with 1x PBS, and anti-CMV IE1 antibody (Millipore Sigma) at 1:1000 dilution in PBS-GC was added. Plates were incubated for 1 hour at 37° C. and cells were washed twice with 1x PBS. Subsequently, AlexaFluor488 goat anti-mouse (Invitrogen) antibody and DAPI stain (ThermoFisher) were added to each well at 1:2000 dilution and 1:1000 dilution in PBS-GC, respectively. The plates were incubated for 1 hour at 37° C. Cells were washed three times with 1x PBS and GFP positive cells were measured using the TargetActivation protocol in High Content Imaging. The number of infected cells were counted, and the IC50 was determined by non-linear regression based on the virus control.

Results: The IC50 values revealed that 26B6 and 23C2 were highly neutralizing for epithelial cells, but as with other mAbs targeting the Uls, 26B6 and 23C2 had no effect in blocking HCMV entry in fibroblasts (Table 9). Among gH-binding antibodies, 2C7 showed the highest potency in neutralizing epithelial cells, around 7- to 32-fold higher potency than the other gH-antibodies tested, while it had similar IC50 in fibroblasts (Table 9). This suggested that the 2C7 targets an epitope that is important for HCMV cell entry in both epithelial cells and fibroblasts.

Neutralizing titers of Pentamer mAbs 26B6, 23C2 and 2C7 are shown in Table 8.

TABLE 8

Neutralizing Titers

| Antibody | IC₅₀ (µg/ml) | | Target |
|---|---|---|---|
| | ARPE-19 | MRC-5 | |
| 916 | 1.92x10⁻⁴ | >10 | ULs |
| 8\|21 | 3.15x10⁻² | >10 | ULs |
| 10P3 | 1.15x10⁻² | >10 | ULs |
| 4\|22 | 6.29x10⁻⁴ | >10 | ULs |
| 23C2 | 3.10x10⁻³ | >10 | ULs |
| 26B6 | 4.07x10⁻³ | >10 | ULs |
| 2C7 | 9.15x10-² | 0.53 | gH |
| 3G16 | 0.64 | 0.11 | gH |
| 13H11 | 2.93 | 0.95 | gH |
| MSL-109 | 0.89 | 0.46 | gH |

TABLE 9

Binding data of 26B6, 23C2 and 2C7 mAbs to Pentamer and gH/gL/gO.

| Pentamer | | | | | gH/gL/gO | | | |
|---|---|---|---|---|---|---|---|---|
| mAb | kₒₙ (1/M_{S}) | k_{off} (1/s) | K_{D} (M) | Rmax (RU) | kₒₙ (1/Ms) | k_{off} (1/s) | K_{D} (M) | Rmax (RU) |
| 23C2 | 2.75 x 10⁵ | 2.85 x 10⁻⁴ | 1.03 x 10⁻⁹ | 258.0 | ns | ns. | ns | 1.4 |
| 26B6 | 4.32 x 10⁵ | 5.30 x 10⁻⁵ | 1.23 x 10⁻¹⁰ | 198.9 | ns | ns | ns | 1.1 |
| 2C7 | 4.47 x 10⁵ | 3.22 x 10⁻⁴ | 7.21 x 10⁻¹⁰ | 287.6 | 4.15 x 10⁵ | 4.16 x 10⁻⁴ | 1.00 x 10⁻⁹ | 212.7 |

### Example 4: Competition Assays with NRP-2 Receptor

Neuropiln-2 (NRP-2) was reported to be the Pentamer receptor for epithelial cells (see e.g., Martinez-Martin et al. Cell 174(5):1158-1171 e1119 (2018)). To investigate whether 26B6, 23C2 and 2C7 inhibit NRP-2 binding to Pentamer, these mAbs were tested in a binding inhibition assay together with a set of known Pentamer mAbs (15D8, 2F4, 10F7, 8121, 10P3, 2C12, 8C15, 916, and 3G16 (see Table 7).

23C2 did not compete with NRP-2, while 26B6 and 2C7 only showed partial inhibition (FIG. X). These data suggest that 23C2 might neutralize HCMV cell entry similarly to 8121 mAb, which also does not compete with NRP-2 binding (Martinez-Martin etal. Cell 174(5): 1158-1171 e1119 (2018)).

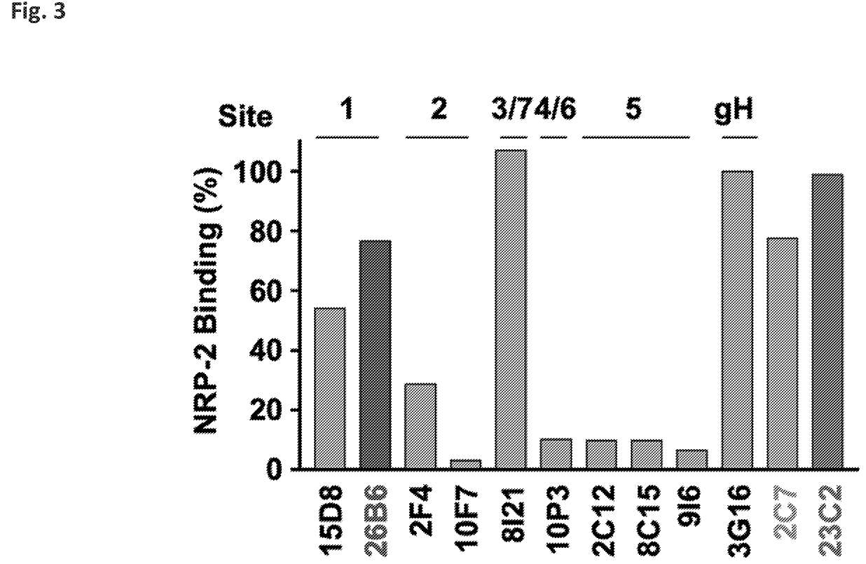

NRP-2 binding assay on HCMV Pentamer was performed using the Octet RED96 instrument (ForteBio Corp). NRP-2, Pentamer and mAb were diluted in 1 × Phosphate Buffered Saline (PBS) with 1% Bovine Serum Albumin (BSA) at the final concentration of 20 µg/ml and the assay was run at 30° C. Biotinylated 3G16 (Biot-3G16) was captured onto Streptavidin biosensors (ForteBio Corp) for 60 seconds in 1 × PBS with 1% BSA. Typical capture levels were between 0.5 and 0.7 nanometers. Biosensor tips were washed for 30 seconds 1 × PBS with 1% BSA before capturing Pentamer for 30 seconds. An additional washing step was done before capturing each Mab. Subsequently, tips preloaded with Biot-3G16/Pentamer/mAb complex were dipped for 200 seconds into NRP-2-containing wells. NRP-2 Binding inhibition was calculated by the following equation: inhibition (%) = 100 -((NRP-2 binding to Pentamer in the presence of a specific mAb) / (secondary NRP-2 binding to pentamer in absence of any mAb) x 100). Results are graphed in FIG. 3, where the bars indicate the percentage of binding of NRP-2 in the presence of the indicated mAb. Each bar represents the average of two independent experiments.

### SEQUENCE LISTING

The amino acid sequences provided herein are shown using standard one- or three-letter abbreviations for amino acids.

SEQ ID NO:1: Heavy Chain variable domain of the 26B6 monoclonal antibody

MEFGLSWVFL VAILEGVHCE VQLVESGGGL VKPGGSLKLS CAASGFTITD YYIYWLRQTP
EKKLEWVAAI SDGGRYTDYP DSVKGRFTMS SDNAKTNLYL QMGSLQSDDT GMYYCARWAS
YGYHWYFDVW GAGTTVTVSS ASTKGPSVFP LAPSSKSTSG GTAALGCLVK DYFPEPVTVS
WNSGALTSGV HTFPAVLQSS GLYSLSSVVT VPSSSLGTQT YICNVNHKPS NTKVDKRVEP
KSCDKTHTCP PCPAPELLGG PSVFLFPPKP KDTLMISRTP EVTCVVVDVS HEDPEVKFNW
YVDGVEVHNA KTKPREEQYN STYRWSVLT VLHQDWLNGK EYKCKVSNKA LPAPIEKTIS
KAKGQPREPQ VYTLPPSREE MTKNQVSLTC LVKGFYPSDI AVEWESNGQP ENNYKTTPPV
LDSDGSFFLY SKLTVDKSRW QQGNVFSCSV MHEALHNHYT QKSLSLSPGK - 470

SEQ ID NO:2: Light Chain variable domain of the 26B6 monoclonal antibody

METPAELLFL LLLWLPDTTG DIQMTQTTSS LSASLGDRVT ISCSASQDIS NYINWYQQKR
DGTVKLLIYD SSSLHSGVPS RFSGSGSGTD YSLTISNLEP EDFATYFCQQ YSNLPPTFGG
GTNLEIKRTV AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN RGEC - 234

SEQ ID NO:3: Heavy Chain variable domain of the 23C2 monoclonal antibody

MEFGLSWVFL VAILEGVHCD VQLQESGPGL VKPSQSLSLT CSVTGYSIKS GYYWNWIRQF
PGNKLEWMGY ISYGGDFTYN PSLKNRISIT RDTSKNQFFL KLNSVTTEDT ATYFCVREPN
WESYFDIWGA GTTVTVSSAS TKGPSVFPLA PSSKSTSGGT AALGCLVKDY FPEPVTVSWN
SGALTSGVHT FPAVLQSSGL YSLSSVVTVP SSSLGTQTYI CNVNHKPSNT KVDKRVEPKS
CDKTHTCPPC PAPELLGGPS VFLFPPKPKD TLMISRTPEV TCVVVDVSHE DPEVKFNWYV
DGVEVHNAKT KPREEQYNST YRWSVLTVL HQDWLNGKEY KCKVSNKALP APIEKTISKA
KGQPREPQVY TLPPSREEMT KNQVSLTCLV KGFYPSDIAV EWESNGQPEN NYKTTPPVLD
SDGSFFLYSK LTVDKSRWQQ GNVFSCSVMH EALHNHYTQK SLSLSPGK - 468

SEQ ID NO:4: Light Chain variable domain of the 23C2 monoclonal antibody

METPAELLFL LLLWLPDTTG DIQMTQSPAS LSASVGETVT ITCRASDNIY SYLTWYHQKQ
GKSPHLLVYN AKTLPEGVPL RFSGTGSGTQ FSLKINSLQP EDFGIYFCLH DYGFPRTFGG
GTILEVKRTV AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN RGEC - 234

SEQ ID NO:5: Heavy Chain variable domain of the 2C7 monoclonal antibody

MEFGLSWVFL VAILEGVHCE VQLQQSGPEL EKPGASVKIS CKASGYSFTG YNMNWVKQSN
GKSLEWIGNI DPYFGGTSYN QKFKGKATLT VDKSSSTAYM ELKSLTSEDS AVYYCARHYG
YFPWFAYWGQ GTLVTVSAAS TKGPSVFPLA PSSKSTSGGT AALGCLVKDY FPEPVTVSWN
SGALTSGVHT FPAVLQSSGL YSLSSVVTVP SSSLGTQTYI CNVNHKPSNT KVDKRVEPKS
CDKTHTCPPC PAPELLGGPS VFLFPPKPKD TLMISRTPEV TCVVVDVSHE DPEVKFNWYV
DGVEVHNAKT KPREEQYNST YRWSVLTVL HQDWLNGKEY KCKVSNKALP APIEKTISKA
KGQPREPQVY TLPPSREEMT KNQVSLTCLV KGFYPSDIAV EWESNGQPEN NYKTTPPVLD
SDGSFFLYSK LTVDKSRWQQ GNVFSCSVMH EALHNHYTQK SLSLSPGK -468

SEQ ID NO:6: Light Chain variable domain of the 2C7 monoclonal antibody

METPAELLFL LLLWLPDTTG DIVLTQSPAS LAVSLGQRAT ISCRASESVD SSGNSFMHWY
QQKPGQPPKL LIYRASNLES GIPARFSGSG SRTDFTLTIN PVGADDVATY YCQQSNEDPW
TFGGGTKLEI KRTVAAPSVF IFPPSDEQLK SGTASVVCLL NNFYPREAKV QWKVDNALQS
GNSQESVTEQ DSKDSTYSLS STLTLSKADY EKHKVYACEV THQGLSSPVT KSFNRGEC -238

SEQ ID NO:7 _HCDR1 of 26B6 mAb, amino acids 50-54 of SEQ ID NO:1

DYYIY

SEQ ID NO:8 _HCDR2 of 26B6 mAb, amino acids 69-85 of SEQ ID NO:1

AISDGGRYTD YPDSVKG

SEQ ID NO:9 _HCDR3 of 26B6 mAb, amino acids 118-129 of SEQ ID NO:1

WASYGYHWYF DV

SEQ ID NO: 10 _LCDR1 of 26B6 mAb, amino acids 44-54 of SEQ ID NO:2

SASQDISNYI N

SEQ ID NO:11 _LCDR2 of 26B6 mAb, amino acids 70-76 of SEQ ID NO:2

DSSSLHS

SEQ ID NO:12 _LCDR3 of 26B6 mAb, amino acids 109-117 of SEQ ID NO:2

QQYSNLPPT

SEQ ID NO:13 _HCDR1 of 23C2 mAb, amino acids 50-55 of SEQ ID NO:3

SGYYWN

SEQ ID NO:14 _HCDR2 of 23C2 mAb, amino acids 70-85 of SEQ ID NO:3

YISYGGDFTY NPSLKN

SEQ ID NO:15 _HCDR3 of 23C2 mAb, amino acids 118-127 of SEQ ID NO:3

EPNWESYFDI

SEQ ID NO:16 _LCDR1 of 23C2 mAb, amino acids 44-54 of SEQ ID NO:4

RASDNIYSYL T

SEQ ID NO:17 _LCDR2 of 23C2 mAb, amino acids 70-76 of SEQ ID NO:4

NAKTLPE

SEQ ID NO:18 _LCDR3 of 23C2 mAb, amino acids 109-117 of SEQ ID NO:4

LHDYGFPRT

SEQ ID NO:19 _HCDR1 of 2C7 mAb, amino acids 50-54 of SEQ ID NO:5

GYNMN

SEQ ID NO:20 _HCDR2 of 2C7 mAb, amino acids 69-85 of SEQ ID NO:5

NIDPYFGGTS YNQKFKG

SEQ ID NO:21 _ HCDR3 of 2C7 mAb, amino acids 118-127 of SEQ ID NO:5

HYGYFPWFAY

SEQ ID NO:22 _LCDR1 of 2C7 mAb, amino acids 44-58 of SEQ ID NO:6

RASESVDSSG NSFMH

SEQ ID NO:23 _LCDR2 of 2C7 mAb, amino acids 74-80 of SEQ ID NO:6

RASNLES

SEQ ID NO:24 _LCDR3 of 2C7 mAb, amino acids 113-121 of SEQ ID NO:6

QQSNEDPWT

SEQ ID NO:25 _Leader of mAb 26B6, 23C2, 2C7 heavy chain variable domain, amino acids 1-19 of SEQ ID NOs: 1, 3, and 5.

MEFGLSWVFL VAILEGVHC

SEQ ID NO:26 _ HFR4 of mAb 26B6 and 23C2 heavy chain variable domain; amino acids 130-140 of SEQ ID NO: 1, amino acids 128-138 of SEQ ID NO: 3.

WGAGTTVTVS S

SEQ ID NO:27 _ Tail of mAb 26B6, 23C2, and 2C7 heavy chain variable domain; amino acids 141-470 of SEQ ID NO: 1, amino acids 139-468 of SEQ ID NO: 3, and amino acids 139-468 of SEQ ID NO: 5

ASTKGPSVFP LAPSSKSTSG GTAALGCLVK DYFPEPVTVS WNSGALTSGV
HTFPAVLQSS GLYSLSSVVT VPSSSLGTQT YICNVNHKPS NTKVDKRVEP
KSCDKTHTCP PCPAPELLGG PSVFLFPPKP KDTLMISRTP EVTCVVVDVS
HEDPEVKFNW YVDGVEVHNA KTKPREEQYN STYRVVSVLT VLHQDWLNGK
EYKCKVSNKA LPAPIEKTIS KAKGQPREPQ VYTLPPSREE MTKNQVSLTC
LVKGFYPSDI AVEWESNGQP ENNYKTTPPV LDSDGSFFLY SKLTVDKSRW
QQGNVFSCSV MHEALHNHYT QKSLSLSPGK

SEQ ID NO:28 _ HFR1 of mAb 26B6 heavy chain variable domain; amino acids 20-49 of SEQ ID NO: 1

EVQLVESGGG LVKPGGSLKL SCAASGFTIT

SEQ ID NO:29 _ HFR2 of mAb 26B6 heavy chain variable domain; amino acids 55-68 of SEQ ID NO: 1

WLRQTPEKKL EWVA

SEQ ID NO:30 _ HFR3 of mAb 26B6 heavy chain variable domain; amino acids 86-117 of SEQ ID NO: 1

RFTMSSDNAK TNLYLQMGSL QSDDTGMYYC AR

SEQ ID NO:31 _ Leader of mAb 26B6 light chain variable domain; amino acids 1-20 of SEQ ID NO: 2

METPAELLFL LLLWLPDTTG

SEQ ID NO:32 _LFR1 of mAb 26B6 light chain variable domain; amino acids 21-43 of SEQ ID NO: 2

DIQMTQTTSS LSASLGDRVT ISC

SEQ ID NO:33 _ LFR2 of mAb 26B6 light chain variable domain; amino acids 55-69 of SEQ ID NO: 2

WYQQKRDGTV KLLIY

SEQ ID NO:34 _ LFR3 of mAb 26B6 light chain variable domain; amino acids 77-108 of SEQ ID NO: 2

GVPSRFSGSG SGTDYSLTIS NLEPEDFATY FC

SEQ ID NO:35 _ LFR4 of mAb 26B6 light chain variable domain; amino acids 118-130 of SEQ ID NO: 2

FGGGTNLEIK RTV

SEQ ID NO:36 _Tail of mAb 26B6 light chain variable domain; amino acids 131-234 of SEQ ID NO: 2

AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN RGEC

SEQ ID NO:37 _ HFR1 of mAb 23C2 heavy chain variable domain; amino acids 20-49 of SEQ ID NO: 3

DVQLQESGPG LVKPSQSLSL TCSVTGYSIK

SEQ ID NO:38 _ HFR2 of mAb 23C2 heavy chain variable domain ; amino acids 56-69 of SEQ ID NO: 3

WIRQFPGNKL EWMG

SEQ ID NO:39 _ HFR3 of mAb 23C2 heavy chain variable domain ; amino acids 86-117 of SEQ ID NO: 3

RISITRDTSK NQFFLKLNSV TTEDTATYFC VR

SEQ ID NO:40 _Leader of mAb 23C2 light chain variable domain; amino acids 1-20 of SEQ ID NO: 4

METPAELLFL LLLWLPDTTG

SEQ ID NO:41 _LFR1 of mAb 23C2 light chain variable domain; amino acids 21-43 of SEQ ID NO: 4

DIQMTQSPAS LSASVGETVT ITC

SEQ ID NO:42 _LFR2 of mAb 23C2 light chain variable domain; amino acids 55-69 of SEQ ID NO: 4

WYHQKQGKSP HLLVY

SEQ ID NO:43 _LFR3 of mAb 23C2 light chain variable domain; amino acids 77-108 of SEQ ID NO: 4

GVPLRFSGTG SGTQFSLKIN SLQPEDFGIY FC

SEQ ID NO:44 _LFR4 of mAb 23C2 light chain variable domain; amino acids 118-130 of SEQ ID NO: 4

FGGGTILEVK RTV

SEQ ID NO:45 _Tail of mAb 23C2 light chain variable domain; amino acids 131-234 of SEQ ID NO: 4

AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN
RGEC

SEQ ID NO:46 _ HFR1 of mAb 2C7 heavy chain variable domain; amino acids 20-49 of SEQ ID NO: 5

EVQLQQSGPE LEKPGASVKI SCKASGYSFT

SEQ ID NO:47 _ HFR2 of mAb 2C7 heavy chain variable domain; amino acids 55-68 of SEQ ID NO: 5

WVKQSNGKSL EWIG

SEQ ID NO:48 _ HFR3 of mAb 2C7 heavy chain variable domain; amino acids 86-117 of SEQ ID NO: 5

KATLTVDKSS STAYMELKSL TSEDSAVYYC AR

SEQ ID NO:49 _HFR4 of mAb 2C7 heavy chain variable domain; amino acids 128-138 of SEQ ID NO: 5

WGQGTLVTVS A

SEQ ID NO:50 _Leader of mAb 2C7 light chain variable domain; amino acids 1-20 of SEQ ID NO: 6

METPAELLFL LLLWLPDTTG

SEQ ID NO:51 _LFR1 of mAb 2C7 light chain variable domain; amino acids 21-43 of SEQ ID NO: 6

DIVLTQSPAS LAVSLGQRAT ISC

SEQ ID NO:52 _LFR2 of mAb 2C7 light chain variable domain; amino acids 59-73 of SEQ ID NO: 6

WYQQKPGQPP KLLIY

SEQ ID NO:53 _LFR3 of mAb 2C7 light chain variable domain; amino acids 81-112 of SEQ ID NO: 6

GIPARFSGSG SRTDFTLTIN PVGADDVATY YC

SEQ ID NO:54 _LFR4 of mAb 2C7 light chain variable domain; amino acids 122-134 of SEQ ID NO: 6

FGGGTKLEI KRTV

SEQ ID NO: 55_Tail of mAb 2C7 light chain variable domain; amino acids 135-238 of SEQ ID NO: 6

AAPSVFIFPP SDEQLKSGTA SVVCLLNNFY PREAKVQWKV DNALQSGNSQESVTEQDSKD STYSLSSTLT LSKADYEKHK VYACEVTHQG LSSPVTKSFN
RGEC

1. An isolated monoclonal antibody or antigen binding fragment thereof, comprising:
(a) a heavy chain variable domain comprising a Heavy Chain Complementarity Determining Region (HCDR) 1 sequence, an HCDR2 sequence, and an HCDR3 sequence, said HCDR sequences from a sequence selected from SEQ ID NO: 1, SEQ ID NO:3, and SEQ ID NO:5 and determined using a method of Kabat, Paratome, Chothia, or IMGT; and
(b) a light chain variable domain comprising a Light Chain Complementarity Determining Region (LCDR) 1 sequence, an LCDR2 sequence, and an LCDR3 sequence, said LCDR sequences from a sequence selected from SEQ ID NO:2, SEQ ID NO:4, and SEQ ID NO:6 and determined using a method of Kabat, Paratome, Chothia, or IMGT;
wherein when said HCDR1, HCDR2, and HCDR3 sequences are from SEQ ID NO:1, said LCDR1, LCDR2, and LCDR3 sequences are from SEQ ID NO: 2;
wherein when said HCDR1, HCDR2, and HCDR3 sequences are from SEQ ID NO:3, said LCDR1, LCDR2, and LCDR3 sequences are from SEQ ID NO: 4; and
wherein when said HCDR1, HCDR2, and HCDR3 sequences are from SEQ ID NO:5, said LCDR1, LCDR2, and LCDR3 sequences are from SEQ ID NO: 6;
and wherein said monoclonal antibody or antigen binding fragment thereof specifically binds to HCMV Pentamer.

2. The isolated monoclonal antibody or antigen binding fragment thereof of claim 1, wherein the HCDR1 sequence comprises SEQ ID NO:7, the HCDR2 sequence comprises SEQ ID NO:8, the HCDR3 sequence comprises SEQ ID NO:9, the LCDR1 sequence comprises SEQ ID NO:10, the LCDR2 sequence comprises SEQ ID NO:11, and the LCDR3 sequence comprises SEQ ID NO:12.

3. The isolated monoclonal antibody or antigen binding fragment thereof of claim 1, wherein the HCDR1 sequence comprises SEQ ID NO:13, the HCDR2 sequence comprises SEQ ID NO:14, the HCDR3 sequence comprises SEQ ID NO:15, the LCDR1 sequence comprises SEQ ID NO:16, the LCDR2 sequence comprises SEQ ID NO:17, and the LCDR3 sequence comprises SEQ ID NO:18.

4. The isolated monoclonal antibody or antigen binding fragment thereof of claim 1, wherein the HCDR1 sequence comprises SEQ ID NO:19, the HCDR2 sequence comprises SEQ ID NO:20, the HCDR3 sequence comprises SEQ ID NO:21, the LCDR1 sequence comprises SEQ ID NO:22, the LCDR2 sequence comprises SEQ ID NO:23, and the LCDR3 sequence comprises SEQ ID NO:24.

5. The isolated monoclonal antibody or antigen binding fragment thereof of claim 1, wherein said monoclonal antibody or antigen binding fragment is humanized.

6. The isolated monoclonal antibody or antigen binding fragment thereof of claim 1, wherein said monoclonal antibody or antigen binding fragment thereof is chimeric.

7. (canceled)

8. (canceled)

9. The isolated monoclonal antibody or antigen binding fragment thereof of claim 1, wherein the antibody or antigen binding fragment thereof is detectably labeled.

10. The isolated monoclonal antibody or antigen binding fragment thereof of claim 9, labeled with a detectable label selected from fluorescent, enzymatic, heavy metal, and radioactive labels.

11. A composition comprising a monoclonal antibody or antigen binding fragment thereof of claim 1, and a pharmaceutically acceptable carrier.

12. (canceled)

13. An isolated nucleic acid molecule encoding the heavy or the light chain variable domain of the monoclonal antibody or antigen binding fragment thereof of claim 1.

14. The isolated nucleic acid molecule of claim 13, operably linked to a promoter.

15. An expression vector comprising the nucleic acid molecule of claim 13.

16. A host cell comprising the expression vector of claim 15.

17. A method of detecting the presence of HCMV Pentamer in a composition, comprising:
(a) obtaining a test sample of the composition;
(b) contacting said test sample with an effective amount of the monoclonal antibody or antigen binding fragment thereof of claim 1 under conditions sufficient to form an immune complex; and
(c) detecting the presence or absence of said immune complex, wherein the presence of the immune complex indicates the presence of HCMV Pentamer in the sample.

18. The method of claim 17, wherein the sample is a biological sample.

19. The method of claim 17, further comprising contacting the sample with a second antibody, wherein said second antibody specifically binds the monoclonal antibody or antigen binding fragment thereof that specifically binds HCMV Pentamer.

20. The method of claim 19, where said second antibody is labeled.

21. A method of detecting the presence of HCMV gH/gL/gO in a composition, comprising:
(a) obtaining a test sample of the composition;
(b) contacting said test sample with an effective amount of the monoclonal antibody or antigen binding fragment of claim 4 under conditions sufficient to form an immune complex; and
(c) detecting the presence or absence of said immune complex, wherein the presence of the immune complex indicates the presence of HCMV gH/gL/gO in the sample.

22. The method of claim 21, wherein the sample is a biological sample.

23. The method of claim 21 , further comprising contacting the sample with a second antibody, wherein said second antibody specifically binds the monoclonal antibody or antigen binding fragment that specifically binds HCMV gH/gL/g0, and wherein said second antibody is labeled.

24. (canceled)

25. (canceled)


SEQUENCE LISTING (55 sequences)

SEQ ID NO: 1 - protein, 470 aa, Artificial Sequence
  source: /note="Description of Artificial Sequence: Synthetic polypeptide"
MEFGLSWVFLVAILEGVHCEVQLVESGGGLVKPGGSLKLSCAASGFTITDYYIYWLRQTPEKKLEWVAAISDGGRYTDYPDSVKGRFTMSSDNAKTNLYLQMGSLQSDDTGMYYCARWASYGYHWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 234 aa, Artificial Sequence
  source: /note="Description of Artificial Sequence: Synthetic polypeptide"
METPAELLFLLLLWLPDTTGDIQMTQTTSSLSASLGDRVTISCSASQDISNYINWYQQKRDGTVKLLIYDSSSLHSGVPSRFSGSGSGTDYSLTISNLEPEDFATYFCQQYSNLPPTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 468 aa, Artificial Sequence
  source: /note="Description of Artificial Sequence: Synthetic polypeptide"
MEFGLSWVFLVAILEGVHCDVQLQESGPGLVKPSQSLSLTCSVTGYSIKSGYYWNWIRQFPGNKLEWMGYISYGGDFTYNPSLKNRISITRDTSKNQFFLKLNSVTTEDTATYFCVREPNWESYFDIWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 234 aa, Artificial Sequence
  source: /note="Description of Artificial Sequence: Synthetic polypeptide"
METPAELLFLLLLWLPDTTGDIQMTQSPASLSASVGETVTITCRASDNIYSYLTWYHQKQGKSPHLLVYNAKTLPEGVPLRFSGTGSGTQFSLKINSLQPEDFGIYFCLHDYGFPRTFGGGTILEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 468 aa, Artificial Sequence
  source: /note="Description of Artificial Sequence: Synthetic polypeptide"
MEFGLSWVFLVAILEGVHCEVQLQQSGPELEKPGASVKISCKASGYSFTGYNMNWVKQSNGKSLEWIGNIDPYFGGTSYNQKFKGKATLTVDKSSSTAYMELKSLTSEDSAVYYCARHYGYFPWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 238 aa, Artificial Sequence
  source: /note="Description of Artificial Sequence: Synthetic polypeptide"
METPAELLFLLLLWLPDTTGDIVLTQSPASLAVSLGQRATISCRASESVDSSGNSFMHWYQQKPGQPPKLLIYRASNLESGIPARFSGSGSRTDFTLTINPVGADDVATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 5 aa, Artificial Sequence
  source: /note="Description of Artificial Sequence: Synthetic peptide"
DYYIY

SEQ ID NO: 8 - protein, 17 aa, Artificial Sequence
  source: /note="Description of Artificial Sequence: Synthetic peptide"
AISDGGRYTDYPDSVKG

SEQ ID NO: 9 - protein, 12 aa, Artificial Sequence
  source: /note="Description of Artificial Sequence: Synthetic peptide"
WASYGYHWYFDV

SEQ ID NO: 10 - protein, 11 aa, Artificial Sequence
  source: /note="Description of Artificial Sequence: Synthetic peptide"
SASQDISNYIN

SEQ ID NO: 11 - protein, 7 aa, Artificial Sequence
  source: /note="Description of Artificial Sequence: Synthetic peptide"
DSSSLHS

SEQ ID NO: 12 - protein, 9 aa, Artificial Sequence
  source: /note="Description of Artificial Sequence: Synthetic peptide"
QQYSNLPPT

SEQ ID NO: 13 - protein, 6 aa, Artificial Sequence
  source: /note="Description of Artificial Sequence: Synthetic peptide"
SGYYWN

SEQ ID NO: 14 - protein, 16 aa, Artificial Sequence
  source: /note="Description of Artificial Sequence: Synthetic peptide"
YISYGGDFTYNPSLKN

SEQ ID NO: 15 - protein, 10 aa, Artificial Sequence
  source: /note="Description of Artificial Sequence: Synthetic peptide"
EPNWESYFDI

SEQ ID NO: 16 - protein, 11 aa, Artificial Sequence
  source: /note="Description of Artificial Sequence: Synthetic peptide"
RASDNIYSYLT

SEQ ID NO: 17 - protein, 7 aa, Artificial Sequence
  source: /note="Description of Artificial Sequence: Synthetic peptide"
NAKTLPE

SEQ ID NO: 18 - protein, 9 aa, Artificial Sequence
  source: /note="Description of Artificial Sequence: Synthetic peptide"
LHDYGFPRT

SEQ ID NO: 19 - protein, 5 aa, Artificial Sequence
  source: /note="Description of Artificial Sequence: Synthetic peptide"
GYNMN

SEQ ID NO: 20 - protein, 17 aa, Artificial Sequence
  source: /note="Description of Artificial Sequence: Synthetic peptide"
NIDPYFGGTSYNQKFKG

SEQ ID NO: 21 - protein, 10 aa, Artificial Sequence
  source: /note="Description of Artificial Sequence: Synthetic peptide"
HYGYFPWFAY

SEQ ID NO: 22 - protein, 15 aa, Artificial Sequence
  source: /note="Description of Artificial Sequence: Synthetic peptide"
RASESVDSSGNSFMH

SEQ ID NO: 23 - protein, 7 aa, Artificial Sequence
  source: /note="Description of Artificial Sequence: Synthetic peptide"
RASNLES

SEQ ID NO: 24 - protein, 9 aa, Artificial Sequence
  source: /note="Description of Artificial Sequence: Synthetic peptide"
QQSNEDPWT

SEQ ID NO: 25 - protein, 19 aa, Artificial Sequence
  source: /note="Description of Artificial Sequence: Synthetic peptide"
MEFGLSWVFLVAILEGVHC

SEQ ID NO: 26 - protein, 11 aa, Artificial Sequence
  source: /note="Description of Artificial Sequence: Synthetic peptide"
WGAGTTVTVSS

SEQ ID NO: 27 - protein, 330 aa, Artificial Sequence
  source: /note="Description of Artificial Sequence: Synthetic polypeptide"
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 30 aa, Artificial Sequence
  source: /note="Description of Artificial Sequence: Synthetic polypeptide"
EVQLVESGGGLVKPGGSLKLSCAASGFTIT

SEQ ID NO: 29 - protein, 14 aa, Artificial Sequence
  source: /note="Description of Artificial Sequence: Synthetic peptide"
WLRQTPEKKLEWVA

SEQ ID NO: 30 - protein, 32 aa, Artificial Sequence
  source: /note="Description of Artificial Sequence: Synthetic polypeptide"
RFTMSSDNAKTNLYLQMGSLQSDDTGMYYCAR

SEQ ID NO: 31 - protein, 20 aa, Artificial Sequence
  source: /note="Description of Artificial Sequence: Synthetic peptide"
METPAELLFLLLLWLPDTTG

SEQ ID NO: 32 - protein, 23 aa, Artificial Sequence
  source: /note="Description of Artificial Sequence: Synthetic peptide"
DIQMTQTTSSLSASLGDRVTISC

SEQ ID NO: 33 - protein, 15 aa, Artificial Sequence
  source: /note="Description of Artificial Sequence: Synthetic peptide"
WYQQKRDGTVKLLIY

SEQ ID NO: 34 - protein, 32 aa, Artificial Sequence
  source: /note="Description of Artificial Sequence: Synthetic polypeptide"
GVPSRFSGSGSGTDYSLTISNLEPEDFATYFC

SEQ ID NO: 35 - protein, 13 aa, Artificial Sequence
  source: /note="Description of Artificial Sequence: Synthetic peptide"
FGGGTNLEIKRTV

SEQ ID NO: 36 - protein, 104 aa, Artificial Sequence
  source: /note="Description of Artificial Sequence: Synthetic polypeptide"
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 30 aa, Artificial Sequence
  source: /note="Description of Artificial Sequence: Synthetic polypeptide"
DVQLQESGPGLVKPSQSLSLTCSVTGYSIK

SEQ ID NO: 38 - protein, 14 aa, Artificial Sequence
  source: /note="Description of Artificial Sequence: Synthetic peptide"
WIRQFPGNKLEWMG

SEQ ID NO: 39 - protein, 32 aa, Artificial Sequence
  source: /note="Description of Artificial Sequence: Synthetic polypeptide"
RISITRDTSKNQFFLKLNSVTTEDTATYFCVR

SEQ ID NO: 40 - protein, 20 aa, Artificial Sequence
  source: /note="Description of Artificial Sequence: Synthetic peptide"
METPAELLFLLLLWLPDTTG

SEQ ID NO: 41 - protein, 23 aa, Artificial Sequence
  source: /note="Description of Artificial Sequence: Synthetic peptide"
DIQMTQSPASLSASVGETVTITC

SEQ ID NO: 42 - protein, 15 aa, Artificial Sequence
  source: /note="Description of Artificial Sequence: Synthetic peptide"
WYHQKQGKSPHLLVY

SEQ ID NO: 43 - protein, 32 aa, Artificial Sequence
  source: /note="Description of Artificial Sequence: Synthetic polypeptide"
GVPLRFSGTGSGTQFSLKINSLQPEDFGIYFC

SEQ ID NO: 44 - protein, 13 aa, Artificial Sequence
  source: /note="Description of Artificial Sequence: Synthetic peptide"
FGGGTILEVKRTV

SEQ ID NO: 45 - protein, 104 aa, Artificial Sequence; identical to SEQ ID NO: 36
  source: /note="Description of Artificial Sequence: Synthetic polypeptide"

SEQ ID NO: 46 - protein, 30 aa, Artificial Sequence
  source: /note="Description of Artificial Sequence: Synthetic polypeptide"
EVQLQQSGPELEKPGASVKISCKASGYSFT

SEQ ID NO: 47 - protein, 14 aa, Artificial Sequence
  source: /note="Description of Artificial Sequence: Synthetic peptide"
WVKQSNGKSLEWIG

SEQ ID NO: 48 - protein, 32 aa, Artificial Sequence
  source: /note="Description of Artificial Sequence: Synthetic polypeptide"
KATLTVDKSSSTAYMELKSLTSEDSAVYYCAR

SEQ ID NO: 49 - protein, 11 aa, Artificial Sequence
  source: /note="Description of Artificial Sequence: Synthetic peptide"
WGQGTLVTVSA

SEQ ID NO: 50 - protein, 20 aa, Artificial Sequence
  source: /note="Description of Artificial Sequence: Synthetic peptide"
METPAELLFLLLLWLPDTTG

SEQ ID NO: 51 - protein, 23 aa, Artificial Sequence
  source: /note="Description of Artificial Sequence: Synthetic peptide"
DIVLTQSPASLAVSLGQRATISC

SEQ ID NO: 52 - protein, 15 aa, Artificial Sequence
  source: /note="Description of Artificial Sequence: Synthetic peptide"
WYQQKPGQPPKLLIY

SEQ ID NO: 53 - protein, 32 aa, Artificial Sequence
  source: /note="Description of Artificial Sequence: Synthetic polypeptide"
GIPARFSGSGSRTDFTLTINPVGADDVATYYC

SEQ ID NO: 54 - protein, 13 aa, Artificial Sequence
  source: /note="Description of Artificial Sequence: Synthetic peptide"
FGGGTKLEIKRTV

SEQ ID NO: 55 - protein, 104 aa, Artificial Sequence; identical to SEQ ID NO: 36
  source: /note="Description of Artificial Sequence: Synthetic polypeptide"